(19) United States
(12) Patent Application Publication
(10) Pub. No.: US 2004/0033544 A1
(43) Pub. Date: Feb. 19, 2004

(54) Polyclonal antibodies against fibrinogen degradation products and associated methods of production and use

(76) Inventor: That T. Ngo, Irvine, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/457,901

(22) Filed: Jun. 9, 2003

Related U.S. Application Data
(63) Continuation-in-part of application No. 09/424,940, filed on Mar. 7, 2000.
(60) Provisional application No. 60/387,179, filed on Jun. 7, 2002.
(60) Provisional application No. 60/445,553, filed on Feb. 7, 2003.

(51) Int. Cl. G01N 33/574; C12P 21/04; C07K 16/30

(52) U.S. Cl. 435/7.23; 435/70.21; 530/388.8

(57) ABSTRACT

Monospecific polyclonal fibrinogen degradation product antibodies, their method of use, the methods to detect cancer and for monitoring the progress of anticancer treatment by immunochemically measuring the quantity of serum FDP in serum are disclosed. The present invention teaches that monospecific polyclonal FDP antibodies that bind to human fibrinogen degradation products (“FDP”) can be obtained by inoculating a laboratory animal with human FDP or human FDP derivatives to induce the production in the inoculated laboratory animal of at least one monospecific polyclonal antibody that binds to human FDP and isolating the monospecific polyclonal antibody. By generating anti-serum to FDP from immunogens and purifying said immunogens using affinity chromatography, increased levels of production of FDP antibodies over the prior art are achieved. A method for screening cancer is disclosed comprising contacting biological sample obtained from a patient with at least one monospecific polyclonal FDP antibody that binds to mammalian FDP. A method is also disclosed for producing a quantitative enzyme linked immunosorbent assay (ELISA) for serum FDP by using monospecific polyclonal antibodies that bind to human FDP.

### RELATED APPLICATIONS

This application claims the benefit of U.S. provisional application serial Nos. 60/387179 filed Jun. 7, 2002 and 60/445553 filed Feb. 7, 2003, and is a continuation-in-part of U.S. patent application Ser. No. 09/424940 filed Mar. 7, 2000, the contents of which are herein incorporated by reference.

### BACKGROUND OF THE INVENTION

1. Field of the Invention

The present invention relates generally to methods of producing and to the production of monospecific polyclonal antibodies against fibrinogen degradation products, to the monospecific polyclonal antibodies themselves and to related methods of use, to the detection of cancers and for monitoring the progress of anticancer treatment by immunochemically measuring the quantity of serum FDP in serum.

2. Description of Related Art

The need for rapid, cost-effective, accurate methods for the identification and diagnosis of cancer has long been felt. Despite recent advances in the understanding of cancer, current techniques for the screening and identification of cancer leave room for improvement. Methods known in the art for screening cancer attempt to detect cancer related antigens by using antibodies. Antibodies are molecules that target and react with pathogens, themselves known as “antigens,” such as viruses and bacteria. The term “antibody” is used in the broadest sense and specifically includes whole bivalent antibody molecules, monovalent Fab fragments, divalent F(ab′)₂ and chemically formed chimeric antibodies, as known in the art, that react with an assortment of protein antigens unique to cancer. Antigens are macromolecules, such as proteins, nucleic acids or polysaccharides, which are capable of eliciting an immune response in the body. The immune systems of mammals and other animals have the ability to detect foreign agents such as antigens associated with cancer and to respond to these antigens by producing antibodies, which specifically target and react with those cancer associated antigenic compounds. Thus, there is a strong correlation between the detection of these cancer associated antigens or the circulating antibodies that target these antigens in a mammal's circulating blood stream and the existence of cancer in that individual. As a result, tests that detect the existence of such antigens or antibodies are useful in the screening and diagnosis of cancer.

Despite strong advances in these antibody mediated testing technologies, known antibody mediated tests do not prove the existence of cancer in a patient. Antibody mediated tests known in the art may produce false positives or may lack sufficient scope to effectively screen for various forms of cancer because they may not identify variants of these target molecules. False positives impose a tremendous detriment to both the individual cancer patient and to society at large. Patients receiving false positives identifying cancer are likely to undergo unnecessary treatments to verify and diagnose their cancers, exacting an enormous emotional and physical toll on the patient, the patient's family and others. Society absorbs the unnecessary costs of false positives in the form of increased medical expenses, the depletion of medical resources and decreases in the productivity of the patients themselves. Antibody tests, which are too specific or narrow to accurately screen for cancer impose a different burden. Failure to accurately screen for cancer may lead to complications in the patient's treatment.

An exemplary method known in the art for such initial cancer screening utilizes blood proteins that circulate in the blood of cancer patients. Identifying the presence of such circulating protein antigens in a patient's blood is known to be a strong initial indication that the patient may have cancer. A well-studied class of such circulating blood antigens indicative of the presence of cancer is fibrinogen degradation products, also referred to as “FDP” (“FDP” is broadly defined as peptide fragments derived from enzymatic degradation of fibrinogen protein). FDP is associated with oncogenic processes, the cellular processes creating cancerous tumors in tissues. Because the presence of circulating FDP antigens is common to all forms of cancer, FDP is known in the art as a “pan-marker” or universal marker antigen useful for the screening of patients to determine if they may have cancer.

As known in the art, patients undergoing physical and medical examinations or suspected of having cancer can have their blood screened for the presence of FDP antigens. After an initial screening for these marker antigens, patients with elevated concentrations of FDP, compared to a “normal,” non-cancerous population, can be further screened to verify if they do in fact have cancer and to identify the specific type of cancer present in their bodies.

Previous methods known in the art for identifying circulating FDP have included the use of serum FDP assays and monoclonal antibodies for use in the assay (determination of the purity of a substance or the amount of any particular constituent of a mixture) of FDP. FDP assays are traditionally performed with antiserum samples, since the assays were usually based on polyclonal antibodies which, in plasma, cross-reacts with the huge excess of fibrinogen, creating an impure composition due to unwanted artifacts. Antiserum is serum that contains a high concentration of antibodies against a particular antigen. As a result, prior art antiserum FDP assays lack specificity and sensitivity due to the impurities caused by this cross-reaction with the excess fibrinogen in the blood. Therefore, antiserum FDP assays may react positively in the absence of cancer. This causes confusion regarding the value of FDP markers, i.e. serum FDP assays produce high rates of false positives.

In response to the ongoing need for more accurate methods to screen cancer, the art has developed monoclonal antibodies for use in the assay of FDP. Monoclonal antibodies very specifically target FDP to react with and hence to identify the presence of FDP antigens circulating in the patient's blood. Monoclonal antibodies used in the assay of FDP do not cross-react with fibrinogen and thus, those assays can be carried out on plasma samples with less risk of false positives.

The use of these prior art monoclonal antibodies targeted against FDP represented a significant advance in the screening of cancer. However, there are aspects to the use of monoclonal FDP antibodies that can be improved. For example, because of the very specific lock-and-key relationship between antibodies and their target antigens, there is a chance that monoclonal antibodies may not produce accurate responses because they might miss individually modified or genetically mutated FDP antigens that are specific to an individual patient. Fibrinogen is known to be heterogeneous in its composition. Further, monoclonal based fibrinogen degradation product assays are challenging to standardized protein because strands known as fibrin in a blood clot, not associated with cancer, may react with the monoclonal antibodies giving a false positive. Further, the possibility remains that monoclonal antibodies against conformational epitopes on native proteins will lose reactivity with antigens that have been minimally perturbed. Further, it takes significantly more time and effort to develop and utilize monoclonal antibodies than polyclonal antibodies.

In summary, due to the heterogeneous nature of human fibrinogen and other complicating factors including the need not to miss potentially significant epitopes of the FDP, and the need to reduce the number of false negatives, there remains a need in the art for more accurate and more reliable methods for the initial or preliminary screening of patients to determine if they may have cancer. Such methods should retain the beneficial specificity of the prior art's monoclonal antibodies against FDP while providing the enhanced ability to identify varying FDP antigens that may be expected in a given patient population. The present invention accomplishes these and other objectives by generating and purifying anti-serum to FDP, creating monospecific polyclonal FDP antibodies.

### SUMMARY OF THE INVENTION

The present invention provides a method for producing a quantitative enzyme-linked immunosorbent assay (ELISA) for serum FDP by using monospecific polyclonal antibodies that bind to human FDP. In one embodiment, the invention provides a method for detecting and quantifying serum FDP in animals using immunochemical method based upon monospecific antibodies to FDP. In a preferred embodiment of the invention, serum FDP is measured using an ELISA. In one embodiment, serum FDP is measured by peroxidase labeled ELISA. In a preferred embodiment, affinity purified anti-FDP antibodies are linked to horseradish peroxidase by use of a crosslinker. In a preferred embodiment, affinity purified anti-FDP antibodies are thiolated and the peroxidase is activated by maleimide derivatives. In a preferred embodiment, the affinity purified anti-FDP antibodies are covalently linked to peroxidase using glutaraldehyde as a crossed linker. A wide variety of chemical methods exist for covalently linking antibodies to enzymes. In a preferred embodiment, the use of hetero-bifunctional crosslinking reagents such as succinimidyl-4-(N-maleimidomethyl) cyclohexane-1-carboxylate (SMCC) or Succinimidyl-m-maleimidobenzoate.

The present invention further provides a method for producing monospecific polyclonal fibrinogen degradation product antibodies that bind to human FDP comprising the steps of inoculating a laboratory animal with prepared immunogens, including human FDP or human FDP derivatives to induce the production of at least one monospecific antibody to FDP and isolating the monospecific antibody to FDP. In one embodiment, affinity chromatography is used to isolate the monospecific polyclonal antibody. In one embodiment, the laboratory animal is a rabbit. In another embodiment, the present invention comprises the monospecific polyclonal FDP antibody that binds to human FDP produced by the steps of inoculating a laboratory animal with prepared immunogens, including human FDP or human FDP derivatives and isolating the monospecific antibody to FDP. In one embodiment, the monospecific polyclonal FDP antibodies are produced using prepared immunogens, including FDP, polymerized FDP and FDP conjugated to keyhole limpet hemocyanin, alone or in-combination.

The present invention further provides a method for the preliminary screening for cancer by taking a biological sample from a patient and contacting the biological sample with at least one monospecific antibody that binds to mammalian FDP. In one embodiment, the mammal is a human.

The present invention further provides a composition useful in screening for cancer, comprising monospecific polyclonal antibody that binds to FDP. In one embodiment, the FDP is human.

The present invention further provides a composition useful for increasing the capacity of FDP to bind FDP antibodies, which comprises FDP coupled to an affinity chromatographic gel. In one embodiment, the affinity chromatographic gel is a polysaccharide gel. In another embodiment, the polysaccharide gel is Sepharose Cl 4B. In another embodiment, the Sepharose Cl 4B is oxidized. In another embodiment, the FDP coupled to oxidized Sepharose Cl 4B produces at least one monospecific polyclonal antibody that binds to FDP.

The present invention further provides monospecific polyclonal antibodies, which target human FDP.

Significant features of the invention include the development of an ELISA by the use of affinity purified polyclonal antibodies to FDP and covalent coupling of the antibodies to enzymes and the use of this enzyme—antibody conjugate together with the same antibodies immobilized onto a solid phase, such as a microtiter well for the detection of cancer and monitoring of treatments.

Significant features of the invention also include increased levels of production of FDP antibodies over the prior art and reduced time and effort in the production of monospecific polyclonal antibodies against FDP versus monoclonal antibodies against FDP. Other significant features of the invention include the use of FDP or its derivatives from human plasma plasmin treated fibrinogen as immunogens to induce the formation of antibodies to FDP in animals and the use of solid supported FDP to obtain monospecific antibodies to FDP by an affinity method. Other features and advantages of the present invention will become apparent from the following detailed description, taken in conjunction with the accompanying drawings.

### BRIEF DESCRIPTION OF THE FIGURES

FIG. 1 is a graphic representation of the effects of the present invention on increasing concentration levels of FDP covalently bound to oxidized Sepharose Cl 4B on the capacity of FDP coupled Sepharose Cl 4B to capture antibodies against FDP.

FIG. 2 is a graphic representation of the titer of antibodies against FDP in rabbits immunized with FDP immunogen expressed as mg antibody per ml of serum plotted against time illustrating the time-course development of the antibody titer in accordance with the teachings of the present invention.

FIG. 3 is a graphic representation of the titer of antibodies against FDP in rabbits immunized with FDP-Hemo-L immunogen expressed as mg antibody per ml of serum plotted against time to show the time-course development of the antibody titer in accordance with the teachings of the present invention.

FIG. 4 is a graphic representation of the titer of antibodies against FDP in rabbits immunized with FDP-Hemo-H immunogen expressed as mg antibody per ml of serum plotted against time to show the time-course development of the antibody titer in accordance with the teachings of the present invention.

FIG. 5 is a graphic representation of the titer of antibodies against FDP in rabbits immunized with FDP-Poly-L immunogen expressed as mg antibody per ml of serum plotted against time to show the time-course development of the antibody titer in accordance with the teachings of the present invention.

FIG. 6 is a graphic representation of the titer of antibodies against FDP in rabbits immunized with FDP-Poly-H immunogen expressed as mg antibody per ml of serum plotted against time illustrating the time-course development of the antibody titer in accordance with the teachings of the present invention.

FIG. 7 is a graphic representation demonstrating the reusability of FDP coupled Sepharose Cl 4B for the isolation of antibodies to FDP in accordance with the teachings of the present invention.

FIG. 8 is a graphic representation of the standard curve for FDP ELISA using conjugate prepared by a method described in Example 19, antibody-coated plate as described in Example 22 and conducted using a procedure described in Example 25.

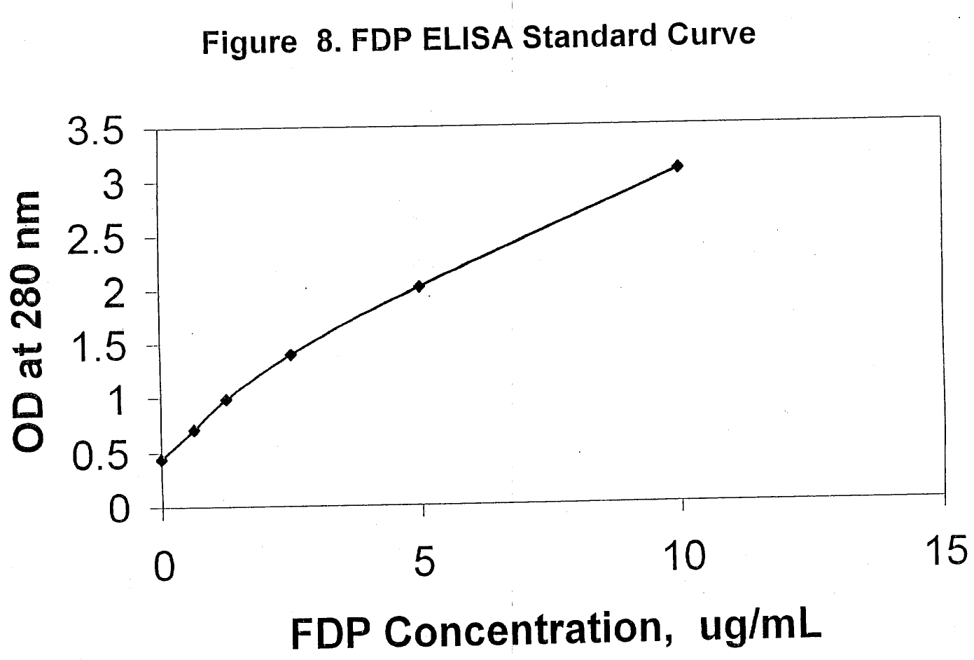

FIG. 9 is a graphic representation of the standard curve for FDP ELISA using conjugate prepared by a method described in Example 20, antibody-coated plate as described in Example 22 and conducted using a procedure described in Example 25.

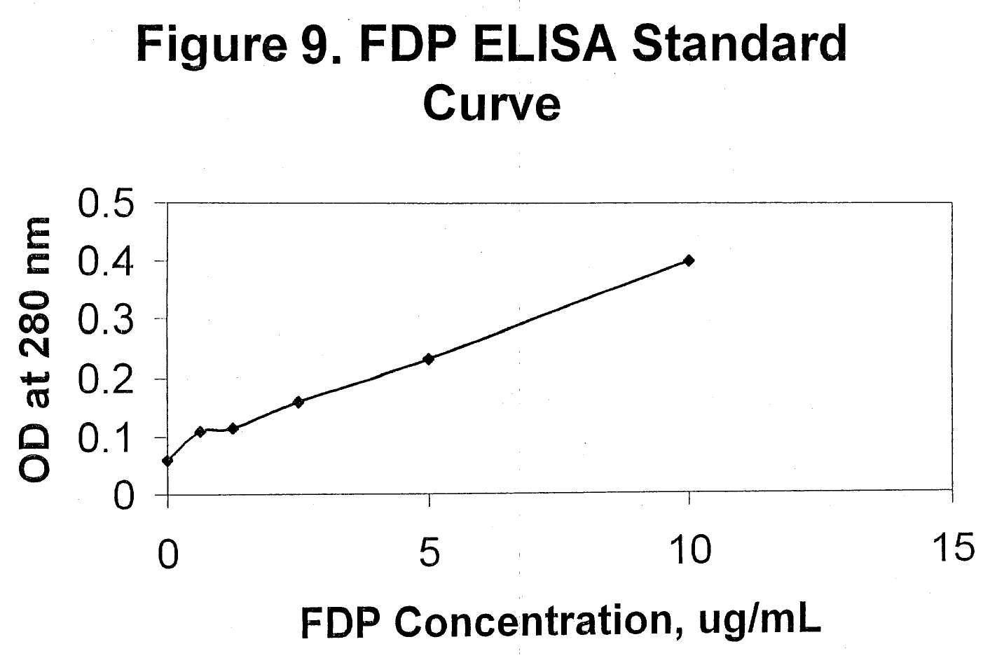

FIG. 10 is a graphic representation of the standard curve for FDP ELISA using conjugate prepared by a method described in Examples 14 and 17, antibody-coated plate as described in Example 22 and conducted using a procedure described in Example 25.

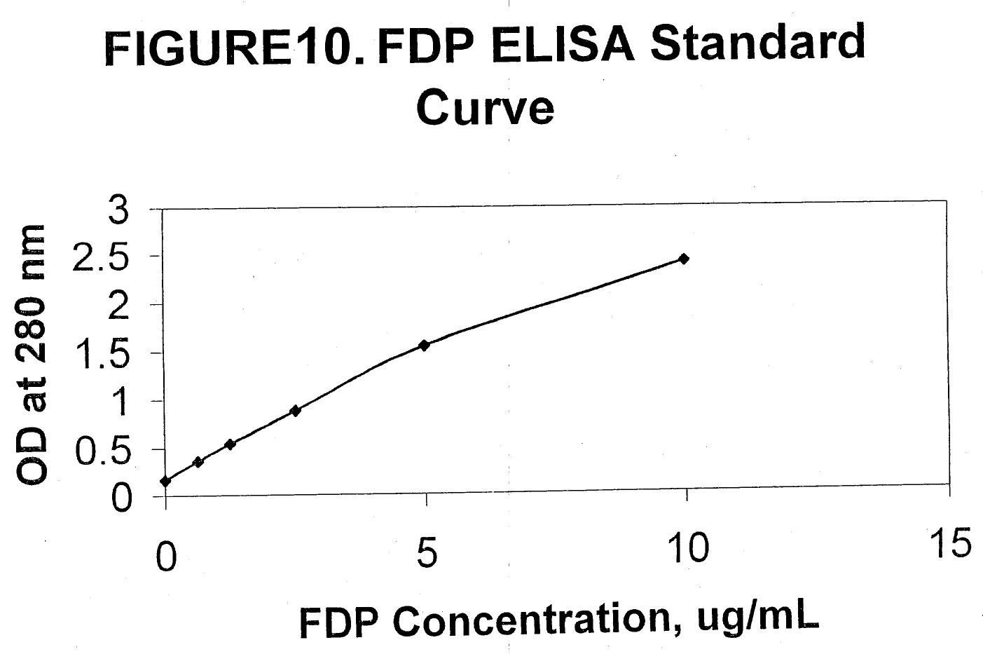

FIG. 11 is a graphic representation of the standard curve for FDP ELISA using conjugate prepared by a method described in Examples 15 and 17, antibody-coated plate as described in Example 22 and conducted using a procedure described in Example 25.

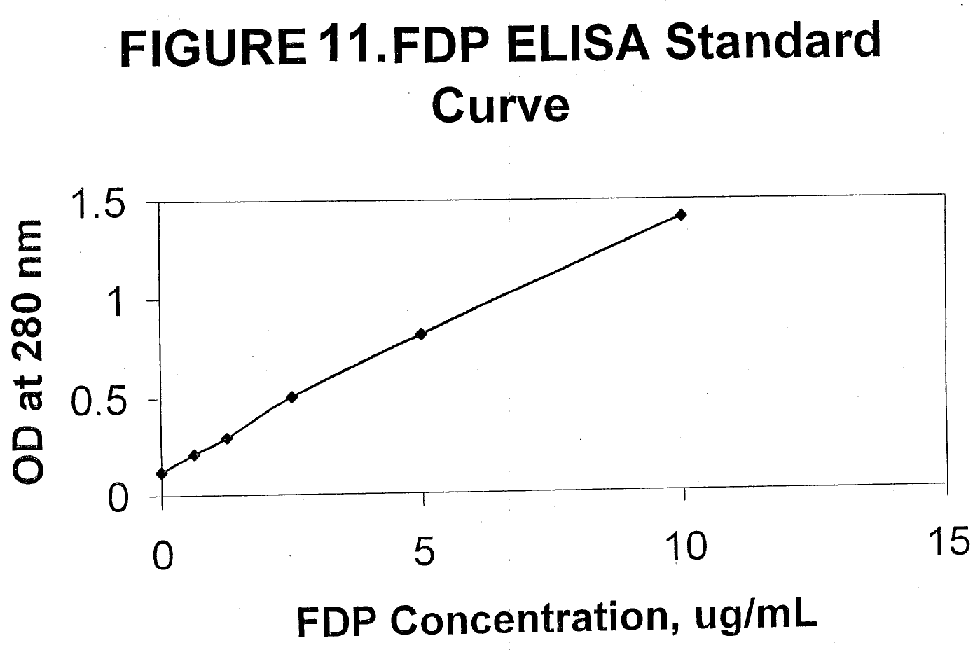

FIG. 12 is a graphic representation of the Receiver-Operator-Characteristic curve for FDP ELISA performed on serum of 43 normal/non-cancer subjects and 20 colon cancer patients. FDP ELISA used in this study consist of microwell plate coated with anti-FDP antibodies and horseradish peroxide labeled anti-FDP antibody conjugate.

FIG. 13 is a graphic representation of the Receiver-Operator-Characteristic curve for FDP ELISA performed on serum of 43 normal/non-cancer subjects and 20 colon cancer patients. FDP ELISA used in this study consist of microwell plate coated with anti-FDP antibodies and horseradish peroxide labeled anti-Fibrinogen antibody conjugate.

FIG. 14 is a graphic representation of the Receiver-Operator-Characteristic curve for FDP ELISA performed on serum of 43 normal/non-cancer subjects and 14 lung cancer patients. FDP ELISA used in this study consist of microwell plate coated with anti-FDP antibodies and horseradish peroxide labeled anti-FDP antibody conjugate.

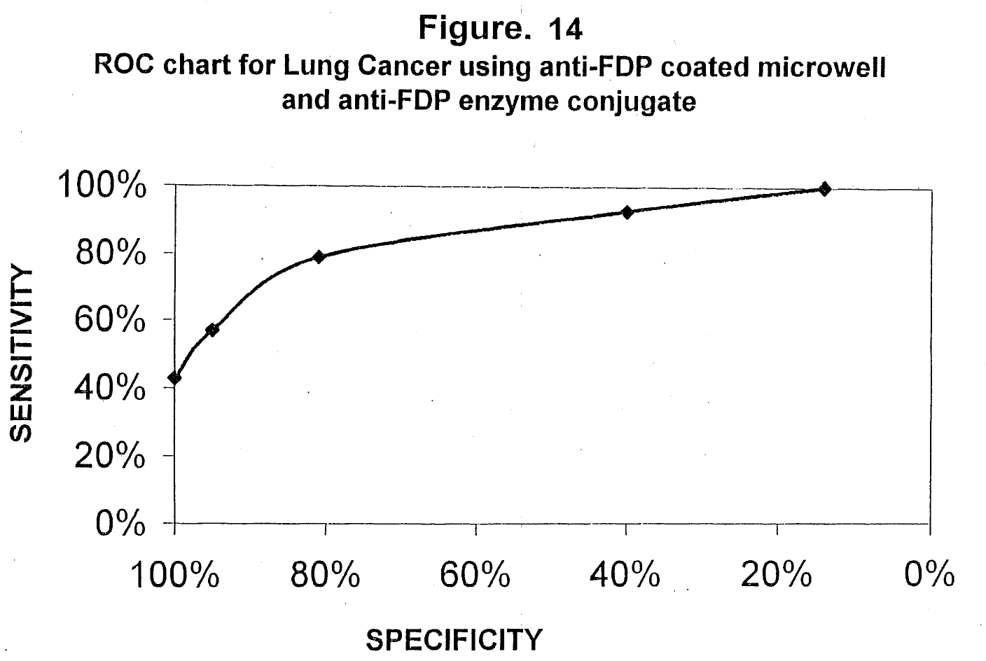

FIG. 15 is a graphic representation of the Receiver-Operator-Characteristic curve for FDP ELISA performed on serum of 43 normal/non-cancer subjects and 14 lung-cancer patients. FDP ELISA used in this study consist of microwell plate coated with anti-FDP antibodies and horseradish peroxide labeled anti-Fibrinogen antibody conjugate.

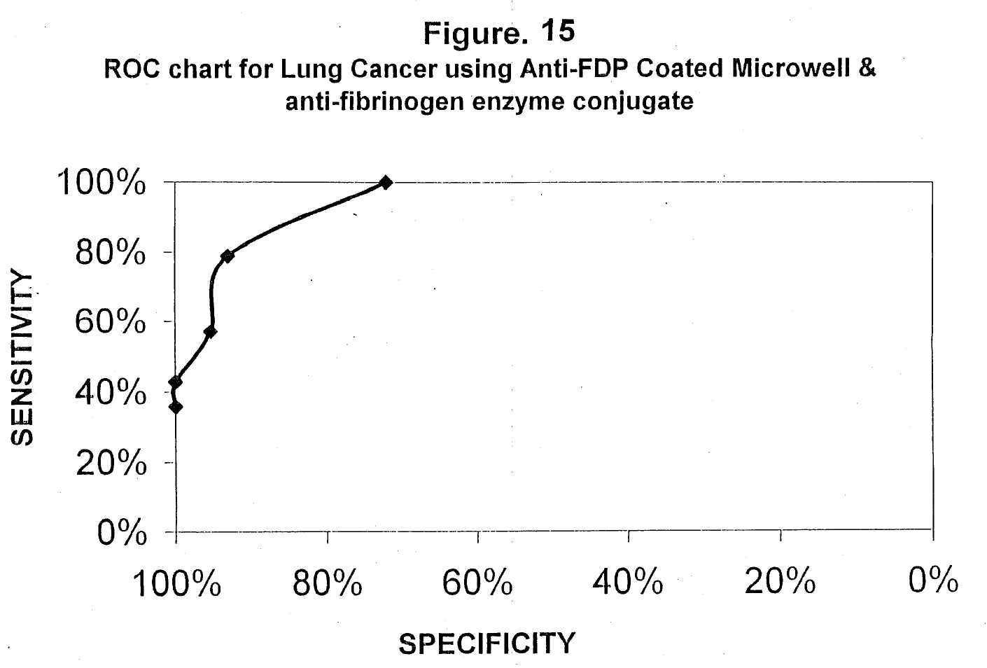

FIG. 16 is a graphic representation of the Receiver-Operator-Characteristic curve for FDP ELISA performed on serum of 43 normal/non-cancer subjects and 20 breast cancer patients. FDP ELISA used in this study consist of microwell plate coated with anti-FDP antibodies and horseradish peroxide labeled anti-FDP antibody conjugate.

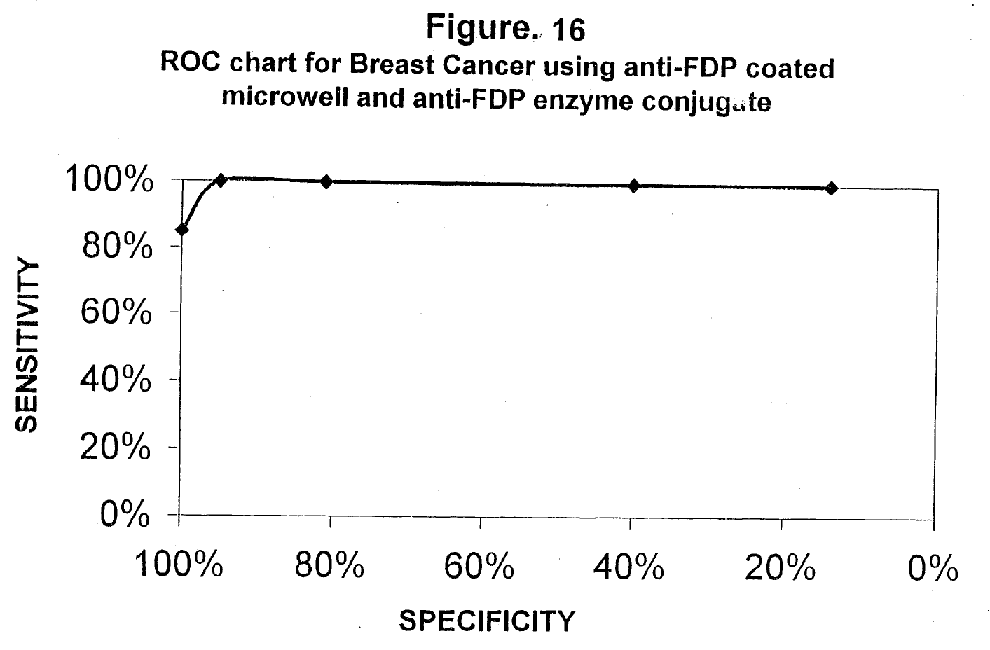

FIG. 17 is a graphic representation of the Receiver-Operator-Characteristic curve for FDP ELISA performed on serum of 43 normal/non-cancer subjects and 20 breast cancer patients. FDP ELISA used in this study consist of microwell plate coated with anti-FDP antibodies and horseradish peroxide labeled anti-Fibrinogen antibody conjugate.

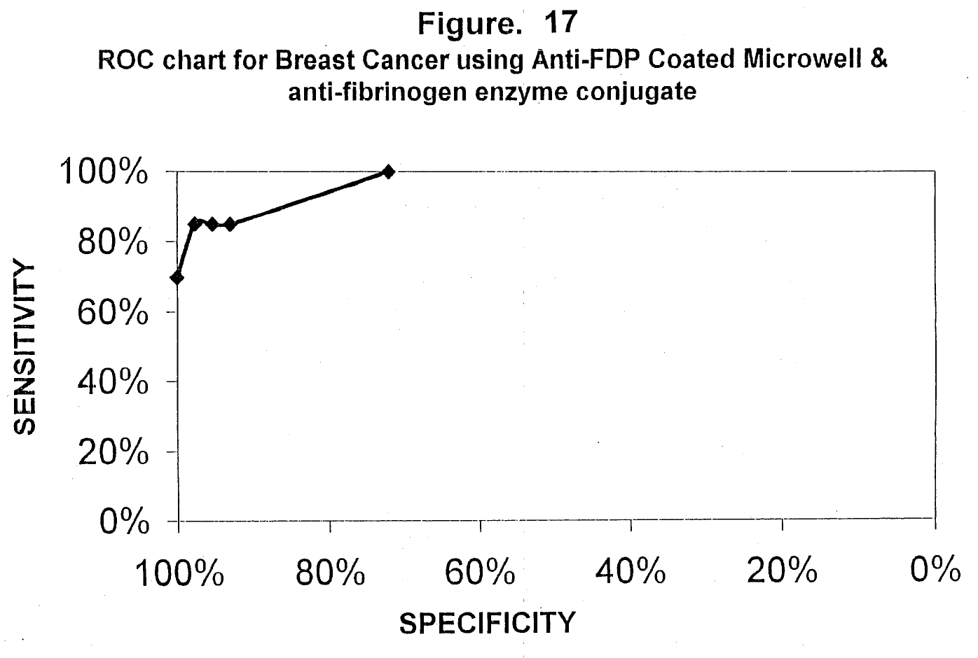

FIG. 18 is a graphic representation of the Receiver-Operator-Characteristic curve for FDP ELISA performed on serum of 43 normal/non-cancer subjects and 20 colon cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-FDP antibody conjugate.

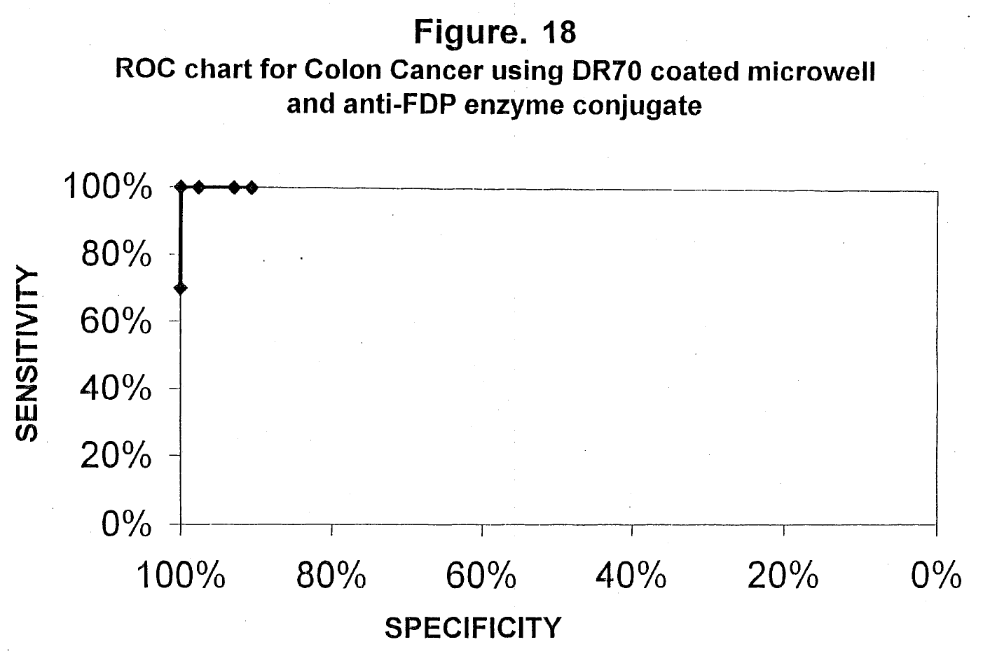

FIG. 19 is a graphic representation of the Receiver-Operator-Characteristic curve for FDP ELISA performed on serum of 43 normal/non-cancer subjects and 20 colon cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-Fibrinogen antibody conjugate.

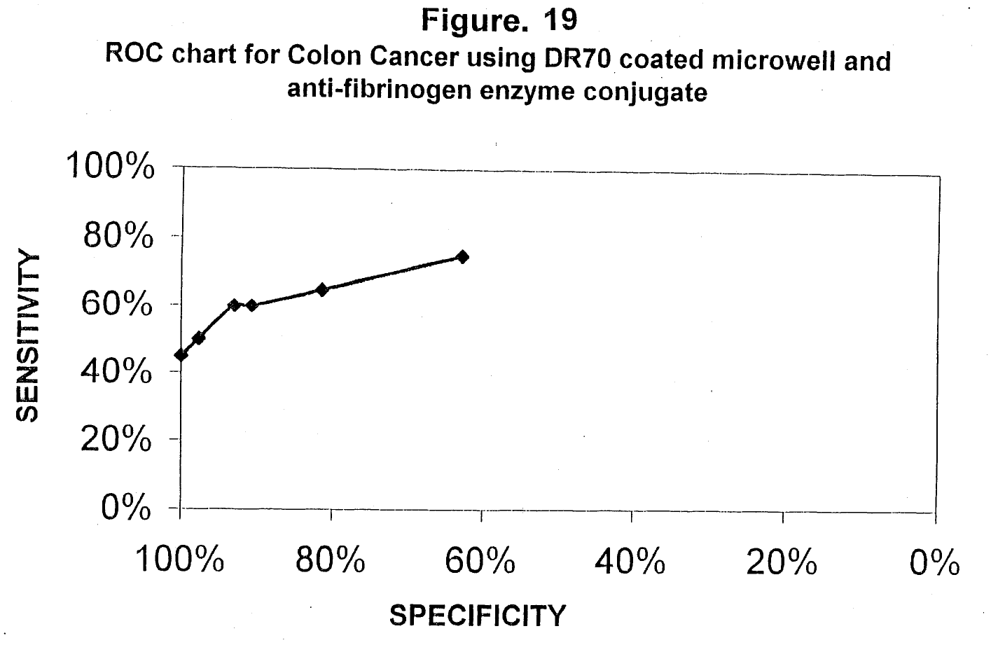

FIG. 20 is a graphic representation of the Receiver-Operator-Characteristic curve for FDP ELISA performed on serum of 43 normal/non-cancer subjects and 14 lung cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-FDP antibody conjugate.

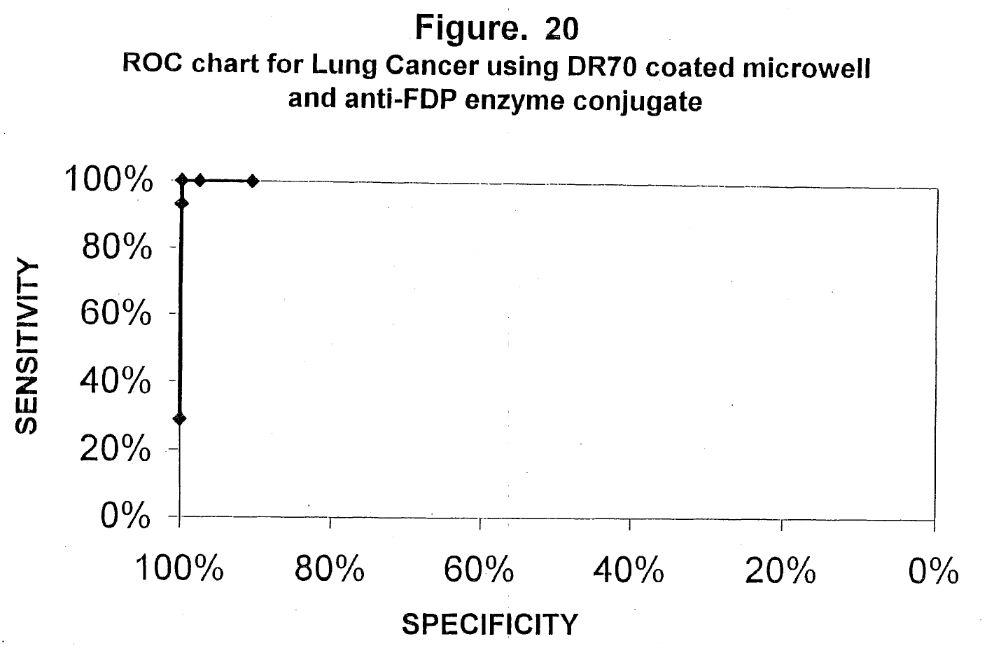

FIG. 21 is a graphic representation of the Receiver-Operator-Characteristic curve for FDP ELISA performed on serum of 43 normal/non-cancer subjects and 14 lung cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-Fibrinogen antibody conjugate.

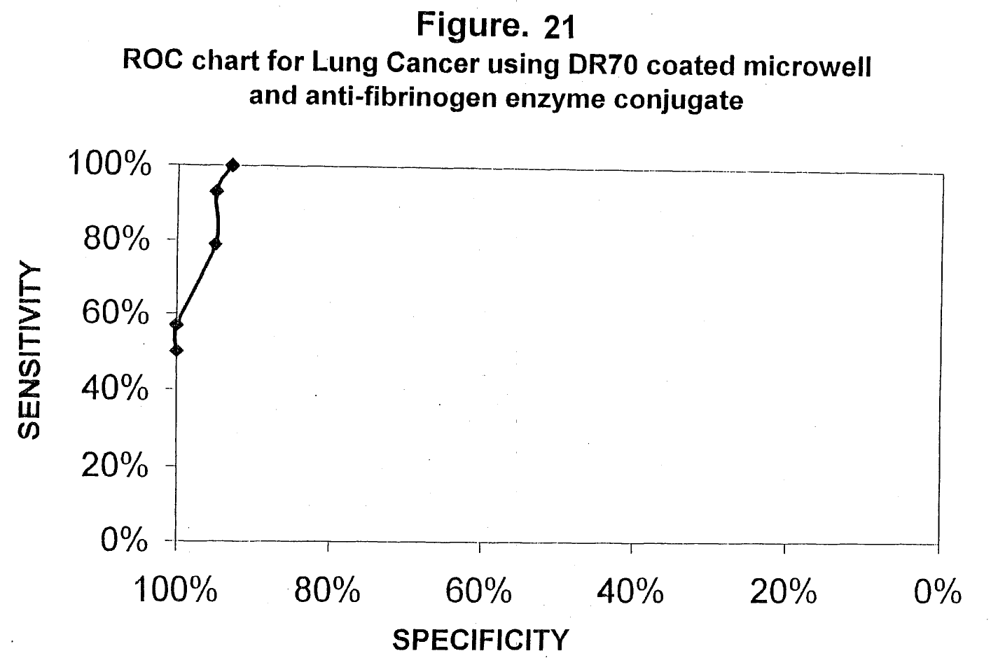

FIG. 22 is a graphic representation of the Receiver-Operator-Characteristic curve for FDP ELISA performed on serum of 43 normal/non-cancer subjects and 20 breast cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-FDP antibody conjugate.

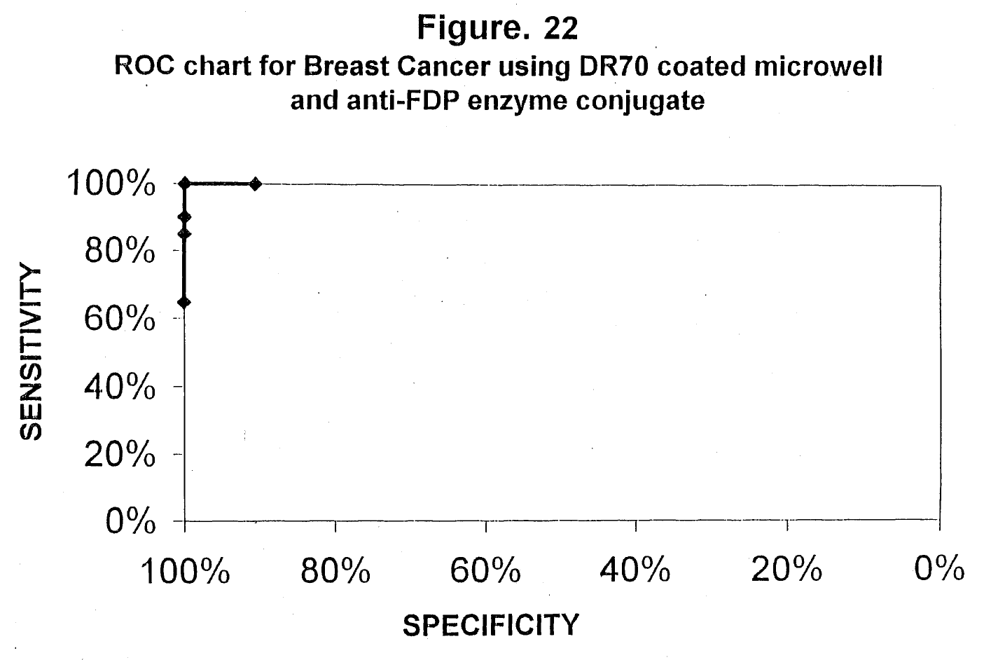

FIG. 23 is a graphic representation of the Receiver-Operator-Characteristic curve for FDP ELISA performed on serum of 43 normal/non-cancer subjects and 20 breast cancer patients. FDP ELISA used in this study consist of microwell plate coated with anti-FDP antibodies isolated from rabbit serum immunized with partially purified antigens from pleural fluid of a lung cancer patient and horseradish peroxide labeled anti-FDP antibody conjugate.

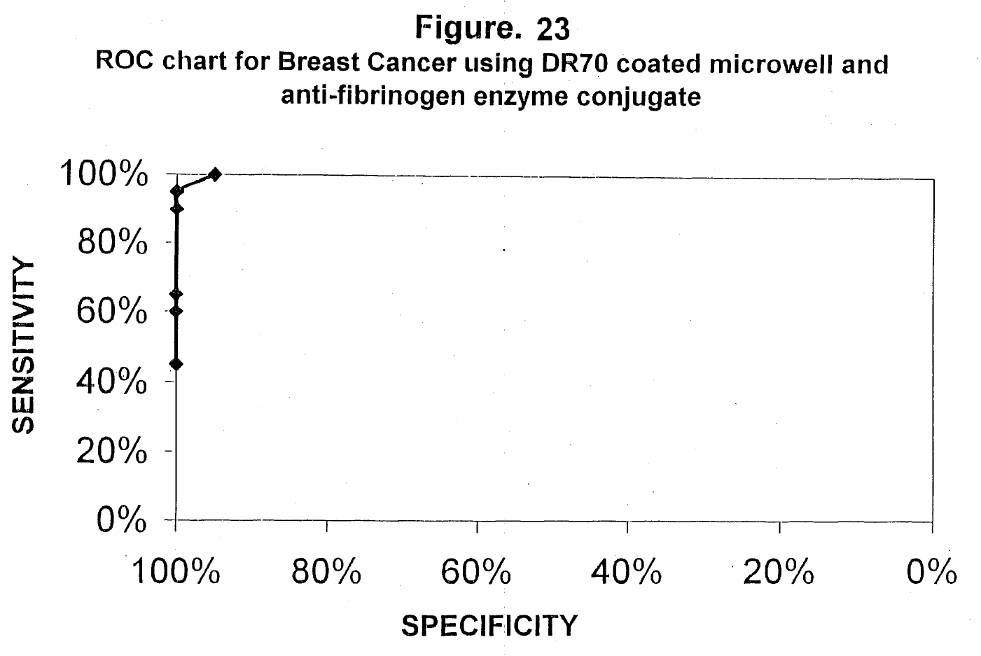

### DETAILED DESCRIPTION OF THE INVENTION

The present invention teaches methods for producing and for the production of monospecific (“monospecific” is specific for only one antigen) polyclonal antibodies against FDP, to the monospecific polyclonal antibodies themselves and to related methods of use. The present invention demonstrates that by generating anti-serum to FDP from immunogens (“immunogens” refers to substances when introduced into the animal body stimulate humoral and cell-mediated immunity) and purifying said immunogens using affinity chromatography (“affinity chromatography” refers to a chemical method for purifying biological substances), increased levels of production of highly purified FDP antibodies over the prior art are achieved.

The present invention teaches that monospecific polyclonal FDP antibodies that bind to human FDP can be obtained by inoculating a laboratory animal with human FDP or human FDP derivatives to induce the production in said inoculated laboratory animal of at least one monospecific polyclonal antibody that binds to human FDP and isolating and purifying the monospecific polyclonal antibody. Affinity chromatography is used to isolate and purify the FDP antibodies obtained from the laboratory animals. In affinity chromatography, a sample containing substances (the target substances) to be purified is contacted to a solid-bound substance (affinity ligand) in conditions that promote strong binding (interaction) with the target substances (such as neutral pH conditions). The bound target substances are released from the solid-bound ligands under conditions (such as acidic or basic conditions) that favor dissociation of the target substances from the solid-phase-bound ligands.

Immunogens can be prepared and injected into laboratory animals. In one embodiment, rabbits can be used. Prior to injecting the laboratory animals with the immunogens, serum should be obtained from each laboratory animal to establish a baseline. The inventor in this case extracted 5-10 milliliters of serum from each of the rabbits.

Immunogens can be prepared from FDP and some FDP derivatives. In this case, the inventor prepared five different immunogens, including: (1) FDP prepared from human fibrinogen digested by human plasma plasmin; (2) FDP-Hemo-L, FDP conjugated to keyhole limpet hemocyanin at low mass ratio (FDP:Hemocyathn=1:5); (3) FDP-Hemo-H, FDP conjugated to keyhole limpet hemocyanin at high mass ratio (FDP:Hemocyanin=2:1); (4) FDP-Poly-L, FDP polymerized with bifunctional crosslinking agents at low FDP concentration; and (5) FDP-Poly-H, FDP polymerized with bifunctional crosslinking agents at high FDP concentration. However, those skilled in the art will recognize that other FDP derivatives may also be used and that other methods may exist for the preparation of the immunogens.

In one embodiment, FDP was prepared from human fibrinogen. Fibrinogen was digested with human plasma plasmin. Human fibrinogen (139 mg) was dissolved in 20 ml MOPS buffer (MOPS:3-[morpholino]propane sulfonic acid, 50 mM; NaCl, 0.1 M; CaCl₂, 2 mM) pH 7.4 at 37° C. Plasmin (5 Units in 1 ml DI water) was added to the fibrinogen solution. The solution was continuously shaken at 37° C. for 3 hours. At the end of 3 hours, the solution was removed from 37° C. and placed on ice.

In another embodiment, FDP-Hemo-L was prepared by linking keyhole limpet hemocyanin (KLH) to lower concentrations of FDP. FDP (8 mg in 1.2 ml) was mixed with 20 mg KLH in 10 ml 0.2 M TAPS (3-[{Tris(hydroxymethyl)methyl}amino]-1-propanesulfonic acid) buffer pH 8.8. Dimethylsuberimidate (12 mg in 0.6 ml ethanol) was added to the above solution of FDP plus KLH and was allowed to react at room temperature for 2 hours. The solution was then dialyzed 2 times at 4° C. against 2 L PBS each time. The volume of the dialysate was measured and divided into 12 aliquots and stored frozen at −40° C.

In a third embodiment, FDP-Hemo-H was prepared by linking keyhole limpet hemocyanin (KLH) to higher concentration of FDP. FDP (25 mg in 3.8 ml) was mixed with 20 mg KLH in 10 ml 0.2 M TAPS (3-[{Tris(hydroxymethyl)methyl}amino]-1-propanesulfonic acid) buffer pH 8.8. Dimethylsuberimidate (DMS, 13 mg in 0.65 ml ethanol) was added to the above solution of FDP plus KLH and was allowed to react at room temperature for 2 hours. The solution was then dialyzed 2 times at 4° C. against 2 L PBS each time. The volume of the dialysate was measured and divided into 12 aliquots and stored frozen at −40° C.

In a fourth embodiment, FDP-Poly-L was prepared by self-polymerization induced by crosslinking with an amino group-specific bifunctional crosslinker DMS. FDP (50 mg in 42.45 ml 0.2 M TAPS pH 8.8) was mixed with 100 mg DMS. The solution reacted at room temperature for 3 hours. Then the solution was dialyzed 2 times at 4° C. against 2 L PBS each time. The volume of the dialysate was measured and divided into 50 aliquots and stored frozen at −40° C.

In a fifth embodiment, FDP-Poly-H was prepared by self-polymerization induced by crosslinking with an amino group-specific bifunctional crosslinker DMS. FDP (50 mg in 7.55 ml 0.2 M TAPS pH 8.8) was mixed with 100 mg DMS. The solution reacted at room temperature for 3 hours. Then the solution was dialyzed 2 times at 4° C. against 2 L PBS each time. The volume of the dialysate was measured and divided into 50 aliquots and stored frozen at −40° C.

After the immunogens are prepared, the laboratory animals should be given an immunization consisting in part of the immunogen. In one embodiment, each rabbit received an injection of an emulsion consisting of 1 mg immunogen in 1 to 1.5 ml phosphate buffer saline and equal volume of Complete Freund' Adjuvant for the first immunization. After an appropriate period of time, the laboratory animals should then be bled and the serum assayed for antibodies against FDP. In one embodiment, three weeks after the injection, each rabbit was bled and the serum was assayed for antibodies against FDP. After an appropriate period of time, the laboratory animals should then be given a booster of an emulsion consisting in part of the immunogen. In one embodiment, one week after the bleeding, booster was given to each rabbit by injection of an emulsion consisting of 1 mg immunogen in to 1.5 ml phosphate buffer saline and equal volume of Incomplete Freund' Adjuvant. The steps of bleeding and assaying serum and giving a booster should be repeated for an appropriate period of time. In one embodiment, the process was repeated for as long as the rabbits survived. When not used, the sera should be stored frozen at approximately −20° C., as was done in this case.

Affinity chromatography is used to isolate and purify the FDP antibodies obtained from the laboratory animals. The monospecific antibodies (antibodies against a single antigen or antigenic determinant) to FDP obtained from the laboratory animals immunized with FDP immunogens are obtained by using FDP immobilized on a solid phase. In one embodiment, in order to couple FDP to the solid phase, Sepharose CL-4B gel, the gel needs to be oxidized to generate aldehydic groups. One skilled in the art will appreciate that other solid phase gels may be used, which may or may not require oxidation. If FDP is coupled to Sepharose CL-4B, the following procedures can be used to oxidize Sepharose Cl-4B: Sepharose Cl-4B (approximately 100 ml) is placed in a sintered glass funnel and the gel is washed approximately 5 times with approximately 100 ml of DI water each time. Sodium meta periodate solution or its equivalent (approximately 0.2M, 100 ml) is added to the washed gel. The gel suspension is wrapped with aluminum foil or its equivalent and shaken at approximately room temperature for approximately 90 minutes. After approximately 90 minutes, about 200 ml of about 0.1 M glycine or its equivalent can be added to the suspension and continuously shaken for about 30 additional minutes to block any remaining aldehydic groups. The gel suspension can be placed in a sintered glass funnel or its equivalent. The gel should be washed approximately 5 times with about 100 ml DI water and approximately 2 times with about 100 ml phosphate buffer saline or its equivalent. Approximately 100 ml of about 0.1% sodium azide solution or its equivalent can be added to the gel and stored at approximately 4° C. In one embodiment, the inventor utilized the foregoing procedure.

After oxidizing the solid phase, the FDP is coupled to the oxidized solid phase by a reductive amination process. The amino-groups of FDP are contacted with the aldehydic groups of the oxidized solid state to form Schiffs bases. In one embodiment, Sepharose Cl 4B is the solid phase. The Schiff's bases are stabilized by sodium borohydride reduction. Oxidized Sepharose Cl 4B is washed with approximately 10 volumes of DI water, 10 volumes of PBS containing 0.5 M NaCl and 10 volumes of PBS. Five aliquots of washed oxidized Sepharose Cl 4B (3 ml each) are each reacted with approximately 0.25 mg, 0.5 mg, 1 mg or 2 mg FDP in 5 ml PBS. To each of the gel suspensions is added approximately 0.5 ml of 0.1 M sodium cyanoborohydride. The suspension is shaken continuously at room temperature for about 8 hours. After approximately 8 hours, about 0.5 ml of 0.1 M glycine in about 0.4 M sodium phosphate buffer pH 7.5 is added to the gel suspension and the gel is shaken for approximately 30 additional minutes. The gel is washed with approximately 10 volumes of DI water, 10 volumes of PBS containing 0.5 M NaCl and 10 volumes of PBS and stored at approximately 4° C.

After the solid state is oxidized and reduced, the titer of antibodies against FDP (“Anti-FDP”) is quantitatively measured using a quantitative affinity chromatographic method (“QAC”). In this QAC method, FDP-coupled to a solid state is used as the affinity gel. In one embodiment Sepharose Cl-4B is used as the affinity gel. The gel is packed into a column and diluted anti-serum obtained from the laboratory animals is passed through the column. In one embodiment the laboratory animals are rabbits. The gel is then washed and eluted with a buffer acid solution. The eluted antibodies against FDP are quantified by reading the absorbance of the antibody solution at 280 nm wavelength in a spectrophotometer. A typical procedure for using QAC method is as follows: (1) FDP coupled Sepharose Cl-4B (1 ml) is packed in 10 ml chromatographic column; (2) the gel is washed sequentially with 10 ml of each of the following solutions: DI water, PBS containing 0.5 M NaCl, PBS containing 0.1M glycine pH 2, DI water and PBS; (3) dilute 6 ml rabbit anti-serum with 6 ml PBS; (4) filter the diluted anti-serum through a 0.2˜filter; (5) load 10 ml of the filtered anti-serum to the column; (6) wash the column with 2 ml PBS, 2 ml PBS containing 0.5M NaCl and 2 ml PBS; (7) elute the antibodies by passing through the column 2 ml PBS containing 0.1M glycine pH 2; (8) collect the elute in a 15 ml conical, graduated centrifuge tube and measure the volume; (9) measure the absorbance of the solution at 280 nm wavelength; and (10) calculate the concentration of antibody isolated as follows Concentration of Antibody Isolated (mg/ml)=Absorbance divided by 1.35. The column can be regenerated for subsequent use by washing with 10 ml each of DI water, PBS containing 0.5 M NaCl, PBS containing 0.1 M glycine pH 2, DI water and PBS.

The present invention further teaches an enzyme-linked immunosorbent assay for the detection of cancer and for monitoring cancer treatment using affinity-purified anti-FDP antibody linked to an enzyme and the same antibodies immobilized onto a solid phase. The affinity-purified anti-FDP antibodies were linked to enzymes by means of cross-linking agents. For the cross-linking of the antibodies and enzymes to take place both of them need to be activated separately by using different activating reagents. Thus, the antibody needs to be thiolated by first reacting with either S-acetylmercaptosuccinic anhydride or with Succinimidyl-S-acetylthioacetate and then reacting with hydroxylamine to obtain free thiol groups by deacetylation. For activating the enzyme, reaction of enzymes with a maleimide bearing heterbifunctional cross-linkers were used. For example, the enzyme can be activated by reacting with Succinimidyl-4-(N-meleimidomethyl)cyclohexane-1-carboxylate to generate maleimide labeled enzyme.

### Thiolation of Anti-FDP IgC

In one embodiment, anti-FDP IgC is thiolated using S-acetylmercaptosuccinic anhydride (SAMSA). Affinity-purified anti-FDP IgG is dialyzed against PBS pH 6.5 at 4° C for 24 hours. Anti-FDP IgC (5 mg, 33 mmol) in PBS pH 6.5 is reacted for 2 hours at room temperature and followed for 20 hours at 4° C. with SAMSA (0.6 mg, 3,450 nmol) dissolved in 0.01 mL N,N-dimethylformamide. The reaction mixture is applied onto a column of Sephadex G-50 fine (1×30 cm), which has been equilibrated with PBS pH6. The SAMSA reacted IgC fractions are pooled. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

In another embodiment, anti-FDP IgC is thiolated using S-acetylmercaptosuccinic anhydride (SAMSA). Affinity-purified anti-FDP IgG is dialyzed against PBS pH 6.5 at 4° C for 24 hours. Anti-FDP IgC (5 mg, 33 nmol) in PBS pH 6.5 is reacted for 2 hours at room temperature and followed for 20 hours at 4° C. with SAMSA (0.6 mg, 3,450 nmol) dissolved in 0.01 mL N,N-dimethylformamide. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

In a third embodiment, anti-FDP IgC is thiolated using S-acetylmercaptosuccinic anhydride (SAMSA). Affinity-purified anti-FDP IgG is dialyzed against PBS pH 6.5 at 4° C. for 24 hours. Anti-FDP IgC (5 mg, 33 nmol) in PBS pH 6.5 is reacted for 2 hours at room temperature with SAMSA (0.6 mg, 3,450 nmol) dissolved in 0.01 mL N,N-dimethylformamide. The reaction mixture is applied onto a column of Sephadex G-50 fine (1×30 cm), which has been equilibrated with PBS pH6. The SAMSA reacted IgC fractions are pooled. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

### Activation of Horseradish Peroxidase with Maleimide Derivative

Horseradish peroxide, HRP (2 mg, 50 nmol) is dissolved in 0.3 mL PBS. pH 7 is reacted with 2,100 nmol of N-succinimidyl-4-(N-maleimidomethyl) cyclohexane-1-carboxylate (SMCC) with vigorous stirring at room temperature for 2 hours and then at 4° C. for 20 hours. The reaction mixture is then centrifuged at room temperature at 2,000 rpm for 15 minutes. The clear supernatant is applied onto a column of Sephadex G-50 fine (1×30 cm), which has been equilibrated with PBS pH6. The maleimide-activated peroxidase fractions were pooled.

In another embodiment, horseradish peroxide, HRP (2 mg, 50 nmol) is dissolved in 0.3 mL PBS. pH 7 is reacted with 2,100 nmol of N-succinimidyl-4-(N-maleimidomethyl) cyclohexane-1-carboxylate (SMCC) with vigorous stirring at room temperature for 2 hours. The reaction mixture is then centrifuged at room temperature at 2,000 rpm for 15 minutes. The clear supernatant is applied onto a column of Sephadex G-50 fine (1×30 cm), which has been equilibrated with PBS pH6. The maleimide-activated peroxidase fractions were pooled.

### Conjugation of Thiolated IcG to Maleimide-Activated Peroxidase

Maleimide-activated peroxidase (2 mg) is incubated with the thiolated ant-FDP IgC (2 mg) in PBS containing 2 M EDTA, pH 6 at room temperature for 2 hours and then at 4° C. for 20 hours. The remaining unreacted thiol groups of the thiolated IgC are deactivated by reacting with 260 nmol N-ethylmaleimide dissolved in 0.05 mL N,N-dimethylformamide at room temperature for 2 hours and at 4° C. for 20 hours.

### Conjugation of Horseradish Peroxidase (HRP) with Anti-FDP IgC Using Glutarldehyde as a Crosslinker

Horseradish peroxidase (5 mg) was dissolved in 0.1 mL of 0.1 M sodium phosphate buffer pH 6.8. 0.01 mL of 25% glutaraldehyde solution is added and allowed to react with HRP at room temperature for 18 hours. The reaction mixture is applied to a column of Sephadex G25F (1×30 cm) that has been equilibrated with the above phosphate buffer. Fractions containing HRP activity are collected and pooled. Glutaraldehyde activated-HRP (2 mg, 50 nmol) is mixed with 2.6 mg of anti-FDP IgC. A solution of 1 M sodium bicarbonate pH 9.5 (1 mL) was added to the mixture of glutaridehyde activated-HRP and anti-FDP IgC. The solution is stirred at 4° C. for 24 hours. 0.05 mL of 0.5 M glycine is added to 0.1 M sodium carbonate pH 9.5 and allowed to react at room temperature for 4 hours. The mixture is applied onto a column of Ultrogel AcA 34 (1.5×48 cm) that has been equilibrated with 0.1 M sodium phosphate pH 6.8. Fractions are collected and the protein content and HRP activity of each fraction is assayed.

### Storge of Peroxidase Anti-FDP IgC Conjugate

Equal volume of stabilzyme (Surmodic Inc.) is mixed with the peroxidase-anti-FDP conjugate, then gentamicin is added as a preservative at 0.1 mg/ml and stored at 4° C.

### Preparation of Anti-FDP Antibody Coated Microtiter Plates

The antibody solution, 120 μL at a concentration of 1.5 μg/mL is added to each microwell and incubated at 25° C. for 12 to 24 hours. Each is washed well 3 times with 170 μL of borate buffer (25 mM sodium borate, 0.1 M boric acid, 0.23 M sodium chloride, 5 mM EDTA, 50 mM 6-aminocaproic acid, pH 8.8). The residual liquid is tapped out by tapping the paper on a clean absorbent paper. 150 μL of 33% Stabilcoat is added and incubated at 25° C. for 12 to 24 hours. The Stabilcoat solution is aspirated and each is washed well 3 times with 170 μL wash buffer (25 mM Tris, 0.1375 M sodium chloride, 2.5 mM M [????] potassium chloride, 0.005% Triton X-100, 0.01% Tween 20, 2.5 mg/L gentamicin, 0.125 mg/L amphotericin B, pH 7.5). The residual liquid is tapped out by tapping the plate on a clean absorbent paper. The plates are placed in a vacuum chamber together with desiccants and vacuum is applied at room temperature for 12 to 24 hours.

The present invention is detailed in the following examples, which are offered by way of illustration and are not intended to limit the invention in any manner. Standard techniques well known in the art or the techniques specifically described below are utilized. All literature references cited in the present specification are hereby incorporated by reference in their entirety.

Commercially available reagents referred to in the examples were used according to manufacturer's instructions unless otherwise indicated.

### EXAMPLES

### Example 1

Preparation of FDP Immunogen

In one embodiment, FDP was prepared from human fibrinogen. Fibrinogen was digested with human plasma plasmin. Human fibrinogen (139 mg) was dissolved in 20 ml MOPS buffer (MOPS:3-[morpholino]propane sulfonic acid, 50 mM; NaCl, 0.1 M; CaCl, 2 mM) pH 7.4 at 37° C. Plasmin (5 Units in l ml DI water) was added to the fibrinogen solution. The solution was continuously shaken at 37° C. for 3 hours. At the end of 3 hours, the solution was removed from 37° C. and placed on ice. Portions of FDP solution were diluted with PBS to 1 mg FDP/ml and were dispensed in 1 ml per vial. The vials were kept frozen until use.

### Example 2

Preparation of Immunogen Consisting of FDP Coupled to Keyhole Limpet Hemocyanin at Low FDP Concentration (FDP-Hemo-L)

In another embodiment, FDP-Hemo-L immunogen was prepared by linking keyhole limpet hemocyanin (KLH) to lower concentrations of FDP. FDP (8 mg in 1.2 ml) was mixed with 20 mg KLH in 10 ml 0.2 M TAPS (3-[{Tris(hydroxymethyl)methyl}amino]-1-propanesulfonic acid) buffer pH 8.8. Dimethylsuberimidate (12 mg in 0.6 ml ethanol) was added to the above solution of FDP plus KLH and was allowed to react at room temperature for 2 hours. The solution was then dialyzed 2 times at 4° C. against 2 L PBS each time. The volume of the dialysate was measured and divided into 12 aliquots and stored frozen at −40° C.

### Example 3

Preparation of Immunogen Consisting of FDP Coupled to Keyhole Limpet Hemocyanin at High FDP Concentration (FDP-Hemo-H)

In another embodiment, FDP-Hemo-H immunogen was prepared by linking keyhole limpet hemocyanin (KLH) to higher concentration of FDP. FDP (25 mg in 3.8 ml) was mixed with 20 mg KLH in 10 ml 0.2 M TAPS (3-[{Tris(hydroxymethyl)methyl}amino]-1-propanesulfonic acid) buffer pH 8.8. Dimethylsuberimidate (DMS, 13 mg in 0.65 ml ethanol) was added to the above solution of FDP plus KLH and was allowed to react at room temperature for 2 hours. The solution was then dialyzed 2 times at 4° C. against 2 L PBS each time. The volume of the dialysate was measured and divided into 12 aliquots and stored frozen at −40° C.

### Example 4

Preparation of Lightly Polymerized FDP (FDP-Poly-L)

In another embodiment, FDP-Poly-L immunogen was prepared by self-polymerization induced by crosslinking with an amino group-specific bifunctional crosslinker DMS. FDP (50 mg in 42.45 ml 0.2 M TAPS pH 8.8) was mixed with 100 mg DMS. The solution reacted at room temperature for 3 hours. Then the solution was dialyzed 2 times at 4° C. against 2 L PBS each time. The volume of the dialysate was measured and divided into 50 aliquots and stored frozen at −40° C.

### Example 5

Preparation of Highly Polymerized FDP (FDP-Poly-H)

In another embodiment, FDP-Poly-H immunogen was prepared by self-polymerization induced by crosslinking with an amino group-specific bifunctional crosslinker DMS. FDP (50 mg in 7.55 ml 0.2 M TAPS pH 8.8) was mixed with 100 mg DMS. The solution reacted at room temperature for 3 hours. Then the solution was dialyzed 2 times at 4° C. against 2 L PBS each time. The volume of the dialysate was measured and divided into 50 aliquots and stored frozen at −40° C.

### Example 6

Preparation of FDP Coupled Sepharose Cl 4B

In another embodiment, Oxidized Sepharose Cl 4B (25 ml) was washed sequentially with 100 ml DI water, 100 ml PBS. The washed and drained gel was mixed with 12.5 ml FDP (100 mg). To this suspension was added 2.5 ml 0.1 M sodium cyanoborohydride in PBS. The suspension was shaken at room temperature for 20 hours. Then 5 ml of 0.1 M glycine in 0.4 M sodium phosphate buffer pH 7.5 was added to the suspension. Continuously shake the suspension for an additional 60 minutes. The gel was centrifuged at room temperature at 1700 rpm for 5 minutes. Save and measure the volume of the supernatant. Measure the absorbance at 280 nm of the supernatant. The gel was then washed with 10 gel volumes of DI water, PBS with 0.5 M NaCl, and PBS. When not in use, the gel was stored in PBS containing 0.1% sodium azide at 4° C.

Based on an extinction coefficient for 1% FDP solution of 5, and the difference between the concentration of FDP in the original solution and the concentration of FDP remaining in the supernatant after the coupling reaction, the amount of FDP coupled to the gel was calculated to be 73.2 mg, which represented a coupling efficiency of 73%.

### Example 7

Effects of the Amount of FDP Coupled to Sepharose Cl 4B on the Quantity of Isolated Antibodies against FDP.

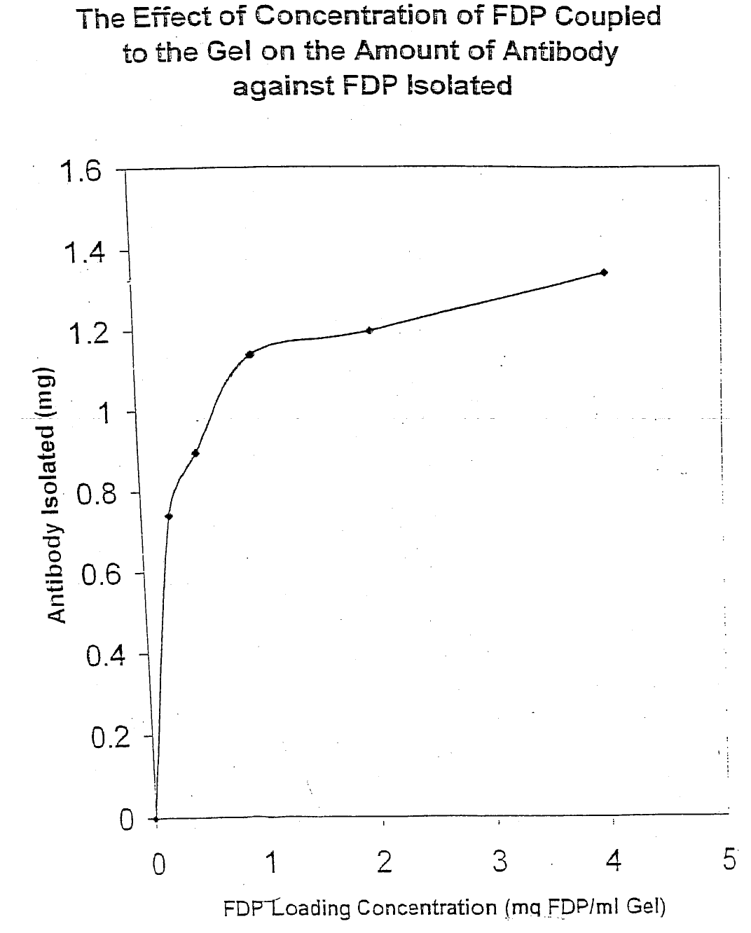

The capacity of FDP coupled Sepharose Cl 4B to capture antibodies against FDP increases with increasing amount of FDP covalently bound to Sepharose Cl 4B. For example, when increasing concentrations of FDP ranging from 0.25 to 4 mg FDP were reacted with one ml of Sepharose Cl 4B, the resulting FDP coupled gels showed their capacity to bind antibodies against FDP increased from 0.74 mg antibody bound per ml gel to 1.34 mg per ml. The relationship between the amount of FDP coupled to Sepharose Cl 4B and the amount of antibodies bound is clearly illustrated in FIG. 1.

### Example 8

Production of Antibodies against FDP in Rabbits Immunized with FDP Immunogen.

Rabbits were immunized with FDP. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, booster was given to each rabbit by injection of an emulsion consisting of 1 mg immunogen in 1 to 1.5 ml phosphate buffer saline and equal volume of Incomplete Freund's Adjuvant.

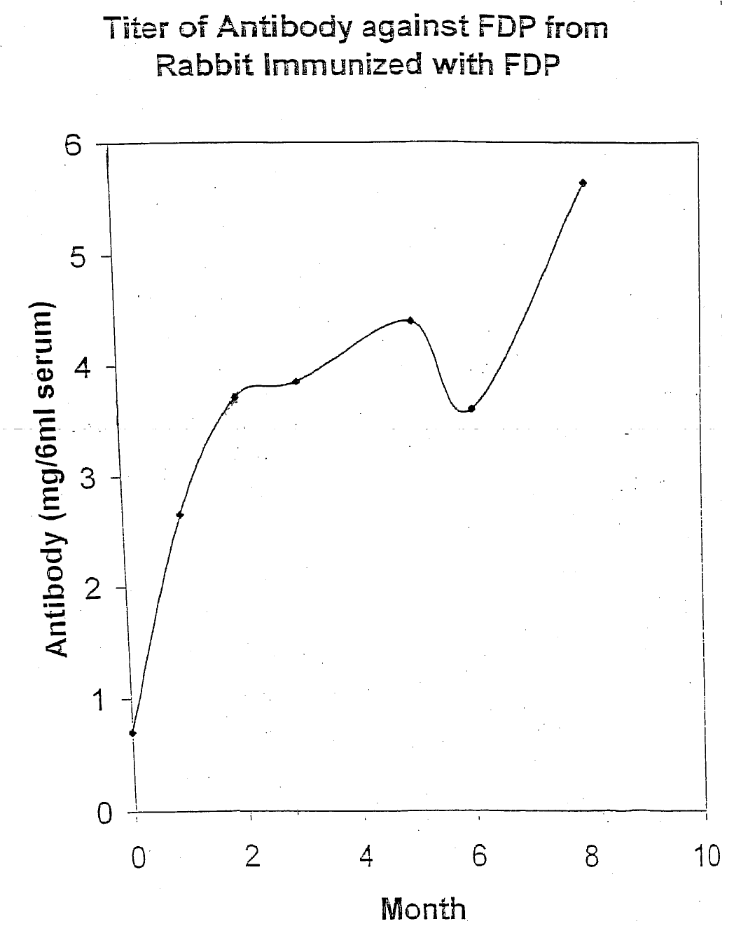

Rabbit serums obtained at various time intervals after immunization were assayed for the concentration of antibodies against FDP by QAC. QAC was carried out as follows: FDP coupled Sepharose Cl-4B (1 ml) was packed in 10 ml chromatographic column. The gel was washed sequentially with 10 ml of each of the following solutions: DI water, PBS containing 0.5M NaCl, PBS containing 0.1M glycine pH 2, DI water and PBS. Six ml rabbit anti-serum was diluted with 6 ml PBS. The diluted anti-serum was filtered through a 0.2μ filter. 10 ml of the filtered anti-serum was loaded to the column. The column was washed with 2 ml PBS, 2 ml PBS containing 0.5M NaCl and 2 ml PBS. The antibodies were eluted by passing through the column 2 ml PBS containing 0.1 M glycine pH 2. The elute was collected in a 15 ml conical, graduated centrifuge tube and measure the volume. The absorbance of the solution was measured at 280 nm wavelength. The concentration of antibody isolated was calculated as follows: Concentration of Antibody Isolated (mg/ml)=Absorbance of the solution divided by 1.35. The titer of antibodies against FDP expressed as mg antibody per ml of serum used was plotted against time to show the time-course of the development of the titer (FIG. 2).

### Example 9

Production of Antibodies against FDP in Rabbits Immunized with FDP-Hemo-L Immunogen.

Rabbits were immunized with FDP-Hemo-L. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, booster was given to each rabbit by injection of an emulsion consisting of 1 mg immunogen in 1 to 1.5 ml phosphate buffer saline and equal volume of Incomplete Freund's Adjuvant.

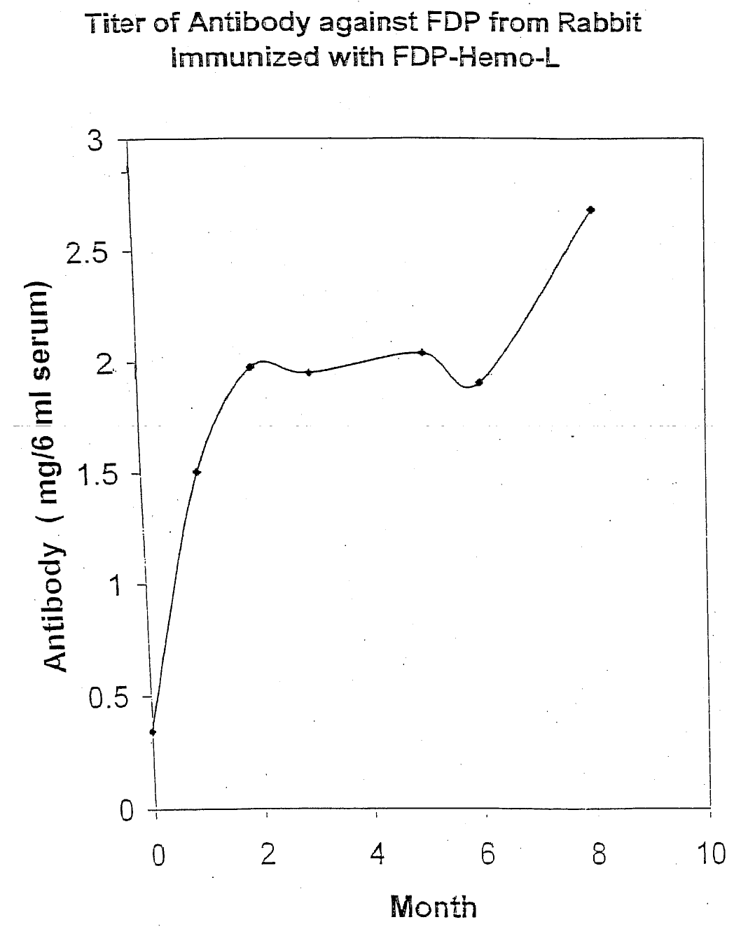

Rabbit serums after immunization at various time intervals were assayed for the concentration of antibodies against FDP by QAC. QAC was carried out as follows: FDP coupled Sepharose Cl-4B (l ml) was packed in 10 ml chromatographic column. The gel was washed sequentially with 10 ml of each of the following solutions: DI water, PBS containing 0.5M NaCl, PBS containing 0.1 M glycine pH 2, DI water and PBS. Six ml rabbit anti-serum was diluted with 6 ml PBS. The diluted anti-serum was filtered through a 0.2μ filter. Ten ml of the filtered anti-serum was loaded to the column. The column was washed with 2 ml PBS, 2 ml PBS containing 0.5M NaCl and 2 ml PBS. The antibodies were eluted by passing through the column 2 ml PBS containing 0.1 M glycine pH 2. The elute was collected in a 15 ml conical, graduated centrifuge tube and the volume was measured. The absorbance of the solution at 280 nm wavelength was measured. The concentration of antibody isolated was calculated as follows: Concentration of Antibody Isolated (mg/ml)=Absorbance of the solution divided by 1.35. The titer of antibodies against FDP expressed as mg antibody per ml of serum was plotted against time to show the time-course of the development of the titer (FIG. 3).

### Example 10

Production of Antibodies against FDP in Rabbits Immunized with FDP-Hemo-H Immunogen.

Rabbits were immunized with FDP-Hemo-H. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, booster was given to each rabbit by injection of an emulsion consisting of 1 mg immunogen in 1 to 1.5 ml phosphate buffer saline and equal volume of Incomplete Freund's Adjuvant.

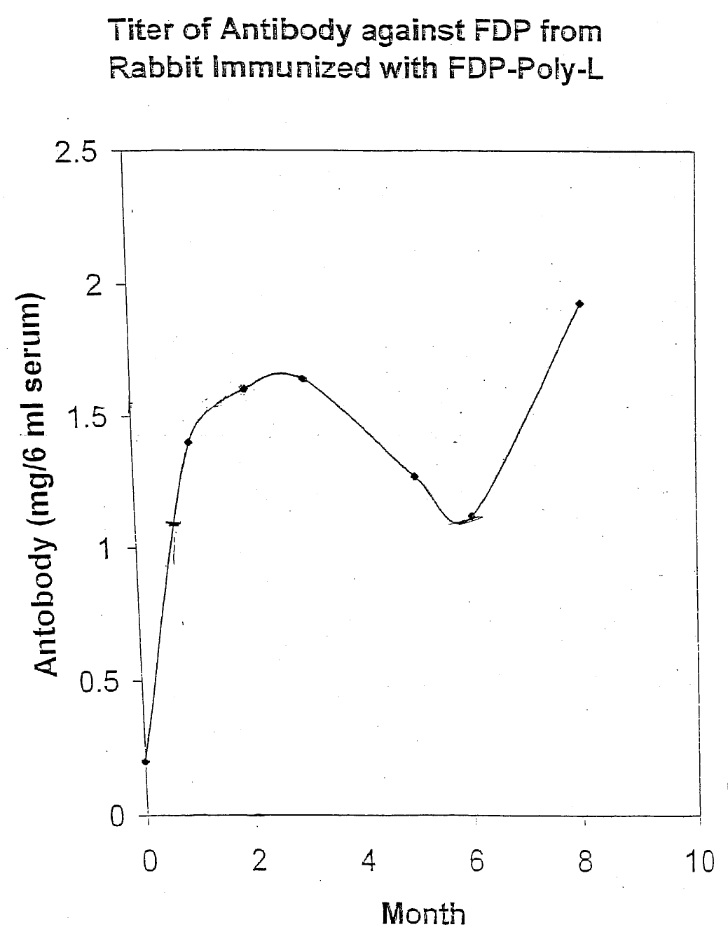

Rabbit serums after immunization at various time intervals were assayed for the concentration of antibodies against FDP by QAC. QAC was carried out as follows: FDP coupled Sepharose Cl-4B (l ml) was packed in 10 ml chromatographic column. The gel was washed sequentially with 10 ml of each of the following solutions: DI water, PBS containing 0.5M NaCl, PBS containing 0.1M glycine pH 2, DI water and PBS. Six ml rabbit anti-serum was diluted with 6 ml PBS. The diluted anti-serum was filtered through a 0.2μ filter. Ten ml of the filtered anti-serum was loaded to the column. The column was washed with 2 ml PBS, 2 ml PBS containing 0.5M NaCl and 2 ml PBS. The antibodies were eluted by passing through the column 2 ml PBS containing 0.1 M glycine pH 2. The elute was collected in a 15 ml conical, graduated centrifuge tube and the volume was measured. The absorbance of the solution at 280 nm wavelength was measured. The concentration of antibody isolated was calculated as follows: Concentration of Antibody Isolated (mg/ml)=Absorbance of the solution divided by 1.35. The titer of antibodies against FDP expressed as mg antibody per ml of serum was plotted against time to show the time-course of the development of the titer (FIG. 4).

### Example 11

Production of Antibodies against FDP in Rabbits Immunized with FDP-Poly-L Immunogen.

Rabbits were immunized with FDP-Poly-L. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, booster was given to each rabbit by injection of an emulsion consisting of 1 mg immunogen in 1 to 1.5 ml phosphate buffer saline and equal volume of Incomplete Freund's Adjuvant.

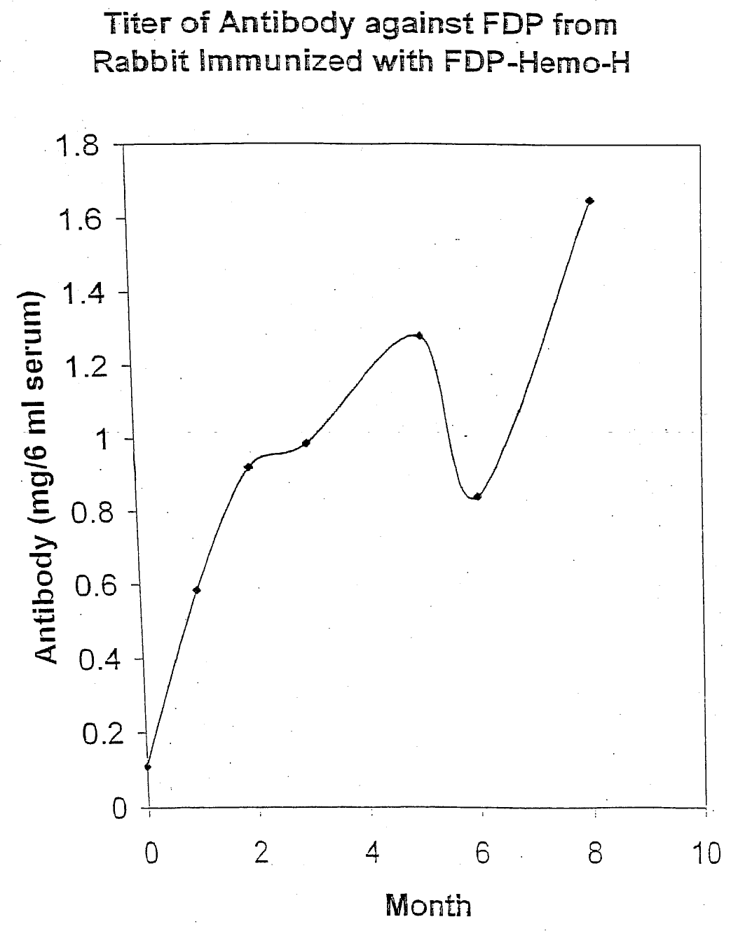

Rabbit serums after immunization at various time intervals were assayed for the concentration of antibodies against FDP by QAC. QAC was carried out as follows: FDP coupled Sepharose Cl-4B (l ml) was packed in 10 ml chromatographic column. The gel was washed sequentially with 10 ml of each of the following solutions: DI water, PBS containing 0.5M NaCl, PBS containing 0.1 M glycine pH 2, DI water and PBS. Six ml rabbit anti-serum was diluted with 6 ml PBS. The diluted anti-serum was filtered through a 0.2μ filter. Ten ml of the filtered anti-serum was loaded to the column. The column was washed with 2 ml PBS, 2 ml PBS containing 0.5M NaCl and 2 ml PBS. The antibodies were eluted by passing through the column 2 ml PBS containing 0.1 M glycine pH 2. The elute was collected in a 15 ml conical, graduated centrifuge tube and the volume was measured. The absorbance of the solution at 280 nm wavelength was measured. The concentration of antibody isolated was calculated as follows: Concentration of Antibody Isolated (mg/ml)=Absorbance of the solution divided by 1.35. The titer of antibodies against FDP expressed as mg antibody per ml of serum was plotted against time to show the time-course of the development of the titer (FIG. 5)

### Example 12

Production of Antibodies against FDP in Rabbits Immunized with FDP-Poly-H Immunogen.

Rabbits were immunized with FDP-Poly-H. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, booster was given to each rabbit by injection of an emulsion consisting of 1 mg immunogen in 1 to 1.5 ml phosphate buffer saline and equal volume of Incomplete Freund's Adjuvant.

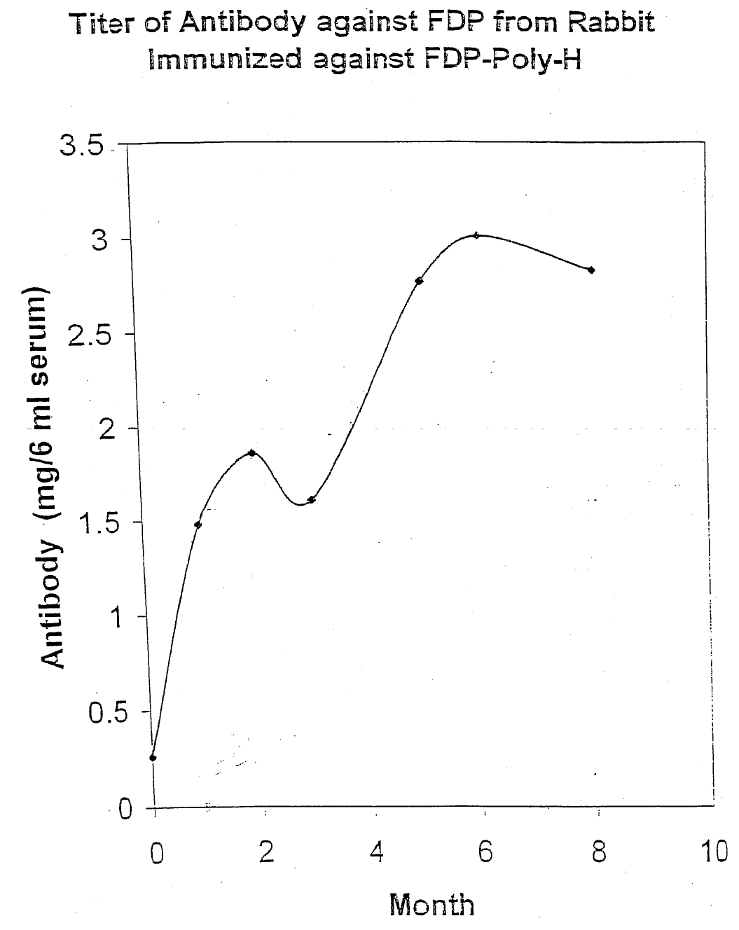

Rabbit serums after immunization at various time intervals were assayed for the concentration of antibodies against FDP by QAC. QAC was carried out as follows: FDP coupled Sepharose Cl-4B (l ml) was packed in 10 ml chromatographic column. The gel was washed sequentially with 10 ml of each of the following solutions: DI water, PBS containing 0.5M NaCl, PBS containing 0.1M glycine pH 2, DI water and PBS. Six ml rabbit anti-serum was diluted with 6 ml PBS. The diluted anti-serum was filtered through a 0.2μ filter. Ten ml of the filtered anti-serum was loaded to the column. The column was washed with 2 ml PBS, 2 ml PBS containing 0.5M NaCl and 2 ml PBS. The antibodies were eluted by passing through the column 2 ml PBS containing 0.1 M glycine pH 2. The elute was collected in a 15 ml conical, graduated centrifuge tube and the volume was measured. The absorbance of the solution at 280 nm wavelength was measured. The concentration of antibody isolated was calculated as follows: Concentration of Antibody Isolated (mg/ml)=Absorbance of the solution divided by 1.35. The titer of antibodies against FDP expressed as mg antibody per ml of serum was plotted against time to show the time-course of the development of the titer (FIG. 6).

### Example 13

Reusability of FDP Coupled Sepharose Cl 4B for Isolation of Antibodies to FDP.

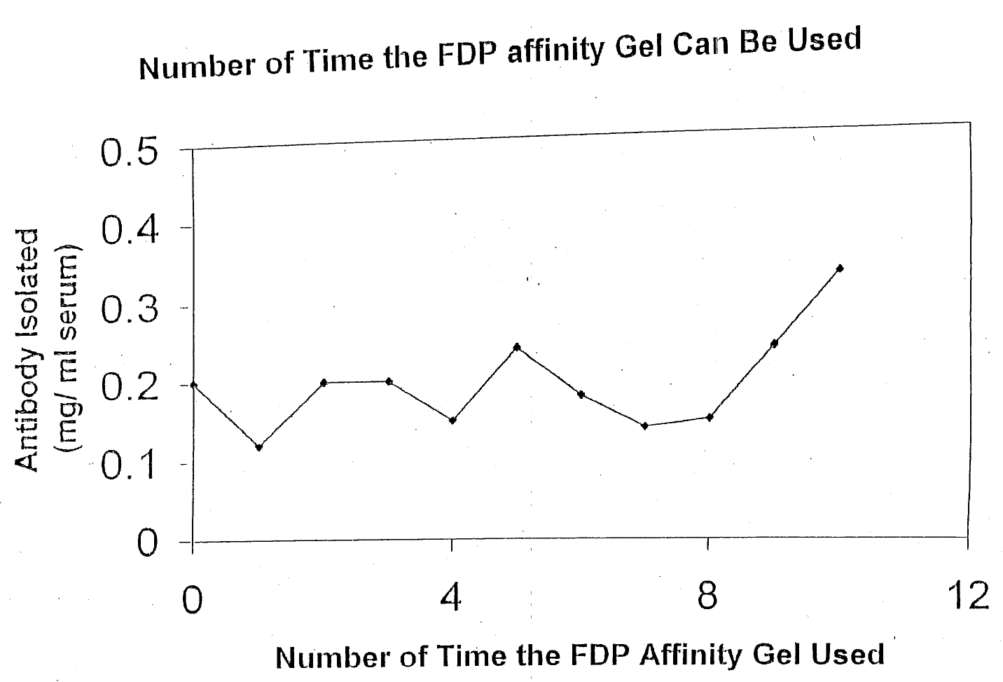

In order to establish the reusability of the FDP coupled gel, a column consisted of packed one ml of the FDP gel was repeatedly used to isolate antibodies against FDP from 12.5 ml of rabbit anti-serum. The results presented in FIG. 7 indicated that after using the column 11 times, no visible trend of column deterioration was observed.

### Example 14

Thiolation of Anti-FDP IgC

In one embodiment, anti-FDP IgC is thiolated using S-acetylmercaptosuccinic anhydride (SAMSA). Affinity-purified anti-FDP IgG is dialyzed against PBS pH 6.5 at 4° C. for 24 hours. Anti-FDP IgC (5 mg, 33 nmol) in PBS pH 6.5 is reacted for 2 hours at room temperature and followed for 20 hours at 4° C. with SAMSA (0.6 mg, 3,450 nmol) dissolved in 0.01 mL N,N-dimethylformamide. The reaction mixture is applied onto a column of Sephadex G-50 fine (1×30 cm), which has been equilibrated with PBS pH6. The SAMSA reacted IgC fractions are pooled. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

### Example 15

Thiolation of Anti-FDP IgC

Anti-FDP IgC is thiolated using S-acetylmercaptosuccinic anhydride (SAMSA). Affinity-purified anti-FDP IgG is dialyzed against PBS pH 6.5 at 4° C. for 24 hours. Anti-FDP IgC (5 mg, 33 nmol) in PBS pH 6.5 is reacted for 2 hours at room temperature and followed for 20 hours at 4° C. with SAMSA (0.6 mg, 3,450 nmol) dissolved in 0.01 mL N,N-dimethylformamide. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

### Example 16

Thiolation of Anti-FDP IgC

Anti-FDP IgC is thiolated using S-acetylmercaptosuccinic anhydride (SAMSA). Affinity-purified anti-FDP IgG is dialyzed against PBS pH 6.5 at 4° C for 24 hours. Anti-FDP IgC (5 mg, 33 nmol) in PBS pH 6.5 is reacted for 2 hours at room temperature with SAMSA (0.6 mg, 3,450 nmol) dissolved in 0.01 mL N,N-dimethylformamide. The reaction mixture is applied onto a column of Sephadex G-50 fine (1×30 cm), which has been equilibrated with PBS pH6. The SAMSA reacted IgC fractions are pooled. SAMSA reacted IgC is added with 0.02 mL of 0.1M EDTA, 0.1 mL of 0.1 M Tris, pH7. The reaction mixture is incubated at room temperature for 5 minutes. The reaction mixture is applied onto a column of Sephadex G-50 fine (1×30 cm), which has been equilibrated with PBS pH6. The thiolated anti-FDP IgC fractions are pooled. The thiolated IgC is reacted immediately with maleimide-activated horseradish peroxide.

### Example 17

Activation of Horseradish Peroxidase with Maleimide Derivative

Horseradish peroxide, HRP (2 mg, 50 nmol) is dissolved in 0.3 mL PBS. pH 7 is reacted with 2,100 nmol of N-succinimidyl-4-(N-maleimidomethyl) cyclohexane-1-carboxylate (SMCC) with vigorous stirring at room temperature for 2 hours and then at 4° C. For 20 hours. The reaction mixture is then centrifuged at room temperature at 2,000 rpm for 15 minutes. The clear supernatant is applied onto a column of Sephadex G-50 fine (1×30 cm), which has been equilibrated with PBS pH6. The maleimide-activated peroxidase fractions were pooled.

### Example 18

Activation of Horseradish Peroxidase with Maleimide Derivative

Horseradish peroxide, HRP (2 mg, 50 nmol) is dissolved in 0.3 mL PBS. pH 7 is reacted with 2,100 nmol of N-succinimidyl-4-(N-maleimidomethyl) cyclohexane-1-carboxylate (SMCC) with vigorous stirring at room temperature for 2 hours. The reaction mixture is then centrifuged at room temperature at 2,000 rpm for 15 minutes. The clear supernatant is applied onto a column of Sephadex G-50 fine (1×30 cm), which has been equilibrated with PBS pH6. The maleimide-activated peroxidase fractions were pooled.

### Example 19

Conjugation of Thiolated IcG to Maleimide-Activated Peroxidase

Maleimide-activated peroxidase (2 mg) is incubated with the thiolated ant-FDP IgC (2 mg) in PBS containing 2 M EDTA, pH 6 at room temperature for 2 hours and then at 4° C. For 20 hours. The remaining unreacted thiol groups of the thiolated IgC are deactivated by reacting with 260 nmol N-ethylmaleimide dissolved in 0.05 mL N,N-dimethylformamide at room temperature for 2 hours and at 4° C. For 20 hours.

### Example 20

Conjugation of Horseradish Peroxidase (HRP) with Anti-FDP IgC Using Glutarldehyde as a Crosslinker

Horseradish peroxidase (5 mg) was dissolved in 0.1 mL of 0.1 M sodium phosphate buffer pH 6.8. 0.01 mL of 25% glutaraldehyde solution is added and allowed to react with HRP at room temperature for 18 hours. The reaction mixture is applied to a column of Sephadex G25F (1×30 cm) that has been equilibrated with the above phosphate buffer. Fractions containing HRP activity are collected and pooled. Glutaraldehyde activated-HRP (2 mg, 50 nmol) is mixed with 2.6 mg of anti-FDP IgC. A solution of 1 M sodium bicarbonate pH 9.5 (1 mL) was added to the mixture of Glutarldehyde activated-HRP and anti-FDP IgC. The solution is stirred at 4° C. For 24 hours. 0.05 mL of 0.5 M glycine is added to 0.1 M sodium carbonate pH 9.5 and allowed to react at room temperature for 4 hours. The mixture is applied onto a column of Ultrogel AcA 34 (1.5×48 cm) that has been equilibrated with 0.1 M sodium phosphate pH 6.8. Fractions are collected and the protein content and HRP activity of each fraction is assayed.

### Example 21

Storage of Peroxidase-Anti-FDP IgC Conjugate

Equal volume of stabilzyme (Surmodic Inc.) is mixed with the peroxidase-anti-FDP conjugate, then gentamicin is added as a preservative at 0.1 mg/ml and stored at 4° C.

### Example 22

Preparation of Anti-FDP Antibody Coated Microtiter Plates

The antibody solution, 120 μL at a concentration of 1.5 μg/mL is added to each microwell and incubated at 25° C. For 12 to 24 hours. Each is washed well 3 times with 170 μL of borate buffer (25 mM sodium borate, 0.1 M boric acid, 0.23 M sodium chloride, 5 mM EDTA, 50 mM 6-aminocaproic acid, pH 8.8). The residual liquid is tapped out by tapping the paper on a clean absorbent paper. 150 μL of 33% Stabilcoat is added and incubated at 25° C. For 12 to 24 hours. The Stabilcoat solution is aspirated and each is washed well 3 times with 170 μL wash buffer (25 mM Tris, 0.1375 M sodium chloride, 2.5 mM M [????] potassium chloride, 0.005% Triton X-100, 0.01% Tween 20, 2.5 mg/L gentamicin, 0.125 mg/L amphotericin B, pH 7.5). The residual liquid is tapped out by tapping the plate on a clean absorbent paper. The plates are placed in a vacuum chamber together with desiccants and vacuum is applied at room temperature for 12 to 24 hours.

### Example 23

Preparation of Diluent Buffer

0.5 mg of Amphotericine B, 0.4 mg aprotinin, 2 mg of gentamicin, 0.4 mg Leupeptin, 0.4 mg Pepsstatin A and 6% Stabilcoat is added to 1L of 40 mM sodium phosphate buffer, pH 7.3. The pH of the solution is adjusted back to 7.3 if necessary.

### Example 24

Preparation of FDP Calibrators

Twenty mL of 5 mM 3-(N-Morpholino) propane-sulfonic acid, pH 7.4 containing 0.1 M sodium chloride and 20 mM calcium chloride (MOPS buffer) is warmed up to 37° C. 139 mg of human fibrinogen is added to the warmed MOPS buffer and is shaken at 37° C. until dissolved. Add 1 mL of DI water to dissolve 5 units of plasmin (from human plasma). The plasmin solution is added to the fibrinogen solution. Contune shaking in 37° C. For 3 hours. 0.4 mL of cocktail protease inhibitors are added to stop the plasmin catalyzed reaction. The solution is diluted to FDP concentrations of 10, 5, 2.5, 1.25, 0.625 μg/mL. The calibrators are stored at 4° C.

### Example 25

Procedure for Performing Enzyme Linked Immunosorbant Assay (ELISA) for FDP

Dilute all serum specimens and calibrators 200 fold with diluent. Place 100 μL of diluted serum or calibrator per microwell. Incubate at room temperature for 30 minutes. Wash microwells 3 to 6 times with 300 μL of wash buffer per microwell each time. Invert the plate and tap it on an absorbent paper. Dispense 100 μL of peroxidase-anti-FDP conjugate to each microwell. Incubate at room temperature for 30 minutes. Wash microwells 3 to 6 times with 300 μL of wash buffer per microwell each time. Dispense 100 μL of peroxidase-anti-FDP conjugate to each microwell. Incubate at room temperature for 15 minutes. Stop the reaction by adding 100 μL of 0.1 M HCL to each microwell.

### Example 26

Preparation of Wash Buffer

Add 3 g of Tris-(hydroxymenthyl) aminomethane, 8 g sodium chloride, 0.2 g Potassium chloride, 50 μL Triton X-100, 0.1 mL Tween 20, 2.5 mg of gentamicin and 1.25 mg Amphotericin B to 0.95 L of deionized water. The pH of the solution is adjusted to 7.5. Add deionized water to 1L.

### Example 27

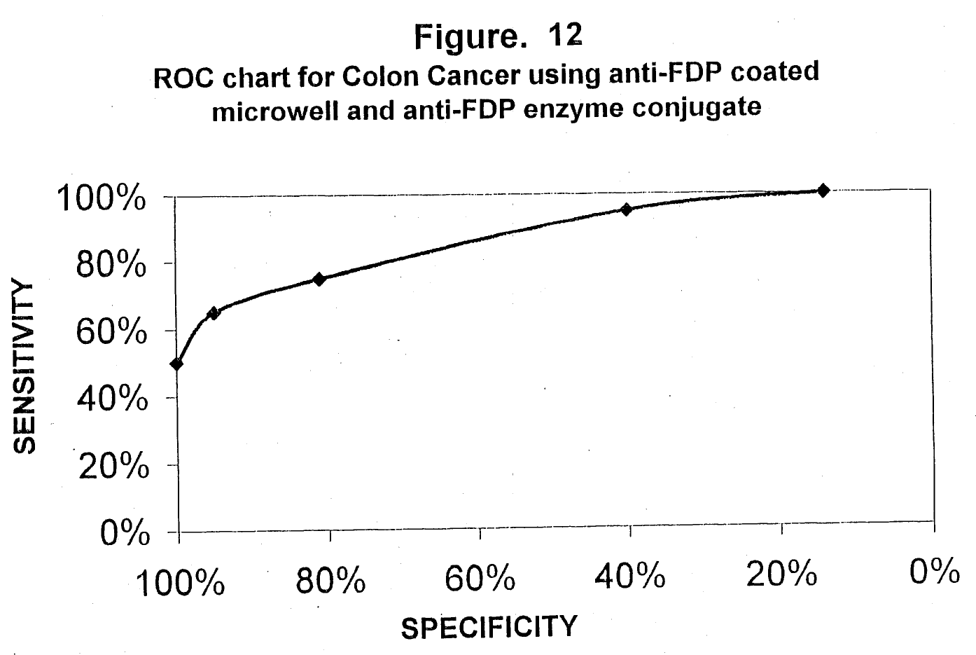

Receiver-Operator-Characteristic Curve for FDP ELISA performed on Serum of 40 normal-non-cancer patients and 20 colon cancer patients. FDP ELISA used in this study consist of microwell plate coated with anti-FDP antibodies and horseradish peroxidase labeled anti-FDP antibody conjugate (See FIG. 12).

### Example 28

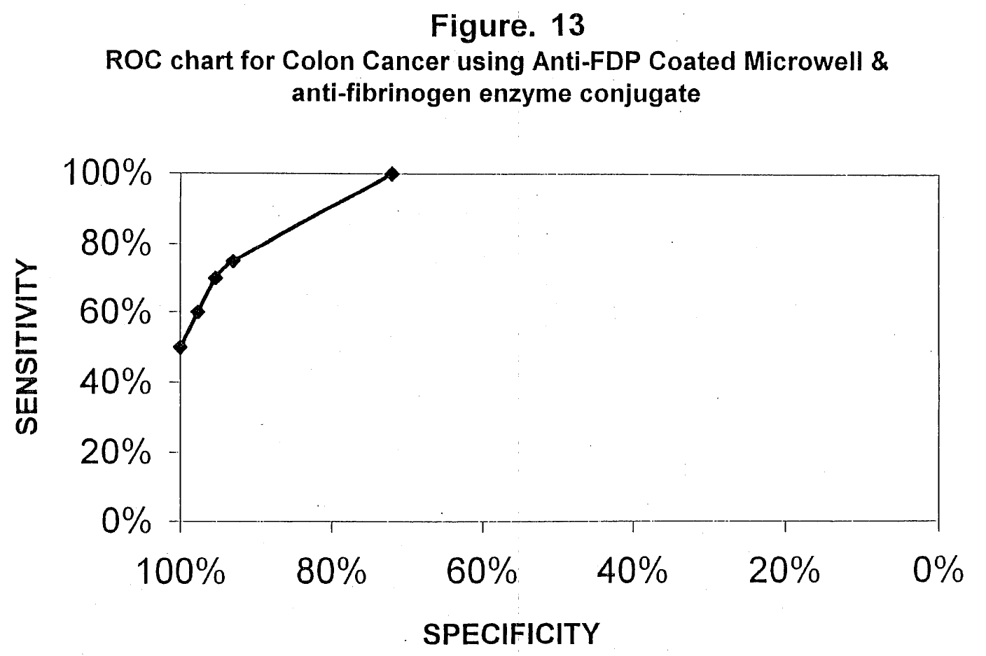

Receiver-Operator-Characteristic Curve for FDP ELISA performed on Serum of 40 normal-non-cancer patients and 20 colon cancer patients. FDP ELISA used in this study consist of microwell plate coated with anti-FDP antibodies and horseradish peroxidase labeled anti-Fibrinogen antibody conjugate (See FIG. 13).

### Example 29

Serum FDP concentration of patients with high and low level PSA. FDP concentration was measured using a sandwich ELISA, consisting of microwell coated with antibodies to FDP and peroxidase labeled antibody to FDP:

TABLE 1

| | Serum FDP conc. | Negative/Positive |
|---|---|---|
| Patient Serum | (ug/ml) | cutoff 3 ug/ml |
| Low PSA | | |
| PSA-L1 | 3.714 | + |
| PSA-L2 | 3.712 | + |
| PSA-L3 | 8.554 | + |
| PSA-L4 | 2.405 | − |
| PSA-L5 | 2.01 | − |
| PSA-L6 | 1.683 | − |
| PSA-L7 | 1.708 | − |
| PSA-L8 | 2.973 | − |
| PSA-L9 | 2.939 | − |
| PSA-L10 | 2.235 | − |
| High PSA | | |
| PSA-H1 | 3.99 | + |
| PSA-H2 | 3.028 | + |
| PSA-H3 | 8.91 | + |
| PSA-H4 | 2.272 | − |
| PSA-H5 | 3.181 | + |
| PSA-H6 | 2.61 | − |
| PSA-H7 | 2.636 | − |
| PSA-H8 | 2.726 | − |
| PSA-H9 | 2.133 | − |
| PSA-H10 | 3.296 | + |

### Example 30

Serum FDP concentration of patients with high and low level PSA. FDP concentration was measured using a sandwich ELISA, consisting of microwell coated with antibodies to FDP and peroxidase labeled antibody to fibrinogen:

TABLE 2

| | Serum FDP conc. | Negative/Positive |
|---|---|---|
| Patient Serum | (ug/ml) | cutoff 2 ug/ml |
| Low PSA | | |
| PSA-L1 | 2.11 | + |
| PSA-L2 | 2.97 | + |
| PSA-L3 | 8.536 | + |
| PSA-L4 | 1.648 | − |
| PSA-L5 | 1.372 | − |
| PSA-L6 | 0.749 | − |
| PSA-L7 | 0.838 | − |
| PSA-L8 | 2.178 | + |
| PSA-L9 | 2.166 | + |
| PSA-L10 | 0.461 | − |
| High PSA | | |
| PSA-H1 | 2.886 | + |
| PSA-H2 | 2.738 | + |
| PSA-H3 | 10.14 | + |
| PSA-H4 | 1.387 | − |
| PSA-H5 | 2.478 | + |
| PSA-H6 | 2.034 | + |
| PSA-H7 | 1.495 | − |
| PSA-H8 | 1.789 | − |
| PSA-H9 | 0.768 | − |
| PSA-H10 | 3.121 | + |

### Example 31

Serum FDP concentration of patients with liver cancer. FDP concentration was measured using a sandwich ELISA, consisting of microwell coated with antibodies to FDP and peroxidase labeled antibody to FDP:

TABLE 3

| | Serum FDP conc. | Negative/Positive |
|---|---|---|
| Patient Serum | (ug/ml) | cutoff 3 ug/ml |
| 1-6 | 3.45 | + |
| 2-8 | 3.241 | + |
| 3-12 | 1.96 | − |
| 4-16 | 3.371 | + |
| 5-22 | 2.676 | − |
| ^{6.32} | 10 | + |
| 7-34 | 10 | + |
| 8-35 | 2.766 | − |
| 9-36 | 6.738 | + |
| 10-45 | 5.172 | + |
| 11-48 | 10 | + |
| 12-51 | 10 | + |
| 13-52 | 10 | + |
| 14-55 | 5.096 | + |
| 15-64 | 2.734 | − |
| 16-65 | 10 | + |
| 17-67 | 6.065 | + |
| 18-79 | 5.342 | + |

### Example 32

Serum FDP concentration of patients with liver cancer. FDP concentration was measured using a sandwich ELISA, consisting of microwell coated with antibodies to FDP and peroxidase labeled antibody to fibrinogen:

TABLE 4

| | Serum FDP conc. | Negative/Positive |
|---|---|---|
| Patient Serum | (ug/ml) | cutoff 3 ug/ml |
| 1-6 | 1.232 | + |
| 2-8 | 1.397 | + |
| 3-12 | 0.623 | − |
| 4-16 | 1.454 | + |
| 5-22 | 0.857 | − |
| 6-32 | 4.493 | + |
| 7-34 | 5.160 | + |
| 8-35 | 0.820 | − |
| 9-36 | 3.021 | + |
| 10-45 | 2.193 | + |
| 11-48 | 5.933 | + |
| 12-51 | 3.103 | + |
| 13-52 | 10.000 | + |
| 14-55 | 2.136 | + |
| 15-64 | 1.203 | + |
| 16-65 | 3.911 | + |
| 17-67 | 2.016 | + |
| 18-79 | 2.315 | + |

### Example 33

Serum FDP concentration of patients with ovarian cancer. FDP concentration was measured using a sandwich ELISA, consisting of microwell coated with antibodies to FDP and peroxidase labeled antibody to FDP:

TABLE 5

| | Serum FDP conc. | Negative/Positive |
|---|---|---|
| Patient Serum | (ug/ml) | cutoff 3 ug/ml |
| O1 | 5.192 | + |
| O2 | 10 | + |
| O3 | 10 | + |
| O4 | 6.471 | + |
| O5 | 10 | + |
| O6 | 10 | + |
| O7 | 3.412 | + |
| O8 | 10 | + |

### Example 34

Serum FDP concentration of patients with ovarian cancer. FDP concentration was measured using a sandwich ELISA, consisting of microwell coated with antibodies to FDP and peroxidase labeled antibody to fibrinogen:

TABLE 6

| | Serum FDP conc. | Negative/Positive |
|---|---|---|
| Patient Serum | (ug/ml) | cutoff 3 ug/ml |
| O1 | 3.869 | + |
| O2 | 10 | + |
| O3 | 10 | + |
| O4 | 5.648 | + |
| O5 | 10 | + |
| O6 | 10 | + |
| O7 | 1.708 | + |
| O8 | 10 | + |

The above description is of one embodiment of the present invention. However, it will be clear to those skilled in the art that various changes and modification may be made without departing from the spirit of the invention.

1. A method for producing monospecific polyclonal fibrinogen degradation product antibodies that bind to human FDP, said method comprising the steps of:
inoculating a laboratory animal with human FDP or human FDP derivatives to induce the production in said inoculated laboratory animal of at least one monospecific polyclonal antibody that binds to human FDP; and
isolating said at least one monospecific polyclonal antibody.

2. The method of claim 1, wherein affinity chromatography is used to isolate said at least one monospecific polyclonal antibody.

3. The method of claim 1 or 2, wherein the laboratory animal is a mammal.

4. The method of claim 1 or 2, wherein the laboratory animal is a rabbit.

5. A composition comprising monospecific polyclonal FDP antibody that binds to human FDP obtained using the method in claim 1.

6. A method for the preliminary screening of a patient for cancer, said method comprising the steps of:
obtaining biological sample from a patient; and
contacting said biological sample with said at least one monospecific polyclonal antibody that binds to mammalian FDP.

7. The method of claim 1 or 2, wherein the mammal is a human.

8. A composition of matter useful in screening for cancer, said composition comprising at least one monospecific polyclonal antibody that binds to FDP.

9. The composition of claim 8 where said FDP is mammalian.

10. The composition of claim 8 where said FDP is human.

11. A composition of matter, useful for increasing the capacity of FDP to bind FDP antibodies, said composition comprising FDP coupled to an affinity chromatographic gel.

12. The composition of claim 11 wherein said affinity chromatographic gel is prepared with a polysaccharide gel.

13. The composition of claim 12 wherein said polysaccharide gel is Sepharose Cl 4B.

14. The composition of claim 13 wherein said Sepharose Cl 4B is oxidized.

15. The composition of claim 11 where said FDP is mammalian.

16. The composition of claim 11 where said FDP is human.

17. A composition of matter useful in screening for cancer, said composition comprising at least one monospecific polyclonal antibody that binds to FDP produced through the steps of claim 11.

18. Monospecific polyclonal antibodies targeting human FDP.

19. A method for the preliminary screening of a patient for cancer, said method comprising the steps of:
thiolating at least one affinity purified anti-FDP antibody;
isolating said at least one affinity purified anti-FDP antibody;
linking said at least one affinity purified anti-FDP antibody to at least one enzyme using cross-linking agents;
immobilizing said at least one affinity purified anti-FDP antibody onto a solid phase;
obtaining biological sample from a patient;
contacting said biological sample with said at least one affinity purified anti-FDP antibody immobilized onto said solid phase; and
measuring serum FDP level.

20. The method of claim 19 wherein the antibodies are affinity purified polyclonal antibodies to FDP.

21. The method of claim 19 wherein the affinity purified polyclonal antibodies to FDP are immobilized onto plastic surface.

22. The method of claim 19 wherein the affinity purified polyclonal antibodies to FDP are labeled with an enzyme.

23. The method of claim 22 wherein the affinity purified polyclonal antibodies to FDP are labeled with horseradish peroxidase.

24. The method of claim 19 wherein the procedure is a sandwich type enzyme labeled immunoassay using microwell coated with antibodies to FDP and horseradish peroxidase conjugated antibodies to FDP.

25. The method of claim 19 wherein the procedure is a sandwich type enzyme labeled immunoassay using microwell coated with antibodies to FDP and horseradish peroxidase conjugated antibodies to fibrinogen.

26. The method of claim 24 is used to screen for lung cancer.

27. The method of claim 24 is used to screen for colon cancer.

28. The method of claim 24 is used to screen for ovarian cancer.

29. The method of claim 24 is used to screen for prostate cancer.

30. The method of claim 24 is used to screen for breast cancer.

31. The method of claim 24 is used to screen for liver cancer.

32. A method for monitoring the treatment of a patient for cancer, said method comprising the steps of:
thiolating at least one affinity purified anti-FDP antibody;
isolating said at least one affinity purified anti-FDP antibody;
linking said at least one affinity purified anti-FDP antibody to at least one enzyme using cross-linking agents;
immobilizing said at least one affinity purified anti-FDP antibody onto a solid phase;
obtaining biological sample from a patient;
contacting said biological sample with said at least one affinity purified anti-FDP antibody immobilized onto said solid phase; and
measuring serum FDP level.

33. The method of claim 32 wherein the antibodies are affinity purified polyclonal antibodies to FDP.

34. The method of claim 32 wherein the affinity purified polyclonal antibodies to FDP are immobilized onto plastic surface.

35. The method of claim 32 wherein the affinity purified polyclonal antibodies to FDP are labeled with an enzyme.

36. The method of claim 35 wherein the affinity purified polyclonal antibodies to FDP are labeled with horseradish peroxidase.

37. The method of claim 32 wherein the procedure is a sandwich type enzyme labeled immunoassay using microwell coated with antibodies to FDP and horseradish peroxidase conjugated antibodies to FDP.

38. The method of claim 32 wherein the procedure is a sandwich type enzyme labeled immunoassay using microwell coated with antibodies to FDP and horseradish peroxidase conjugated antibodies to fibrinogen.

39. The method of claim 37 is used to monitor the treatment of lung cancer.

40. The method of claim 37 is used to monitor the treatment of colon cancer.

41. The method of claim 37 is used to monitor the treatment of ovarian cancer.

42. The method of claim 37 is used to monitor the treatment of prostate cancer.

43. The method of claim 37 is used to monitor the treatment of breast cancer.

44. The method of claim 37 is used to monitor the treatment of liver cancer.

45. A composition of matter useful for the detection of cancer comprising an enzyme-linked immunosorbent assay.

46. A composition of matter useful for the monitoring of cancer treatment comprising an enzyme-linked immunosorbent assay.

47. A method for producing an enzyme-linked immunosorbent assay for the detection of cancer, said method comprising the steps of:
thiolating at least one affinity purified anti-FDP antibody;
isolating said at least one affinity purified anti-FDP antibody;
linking said at least one affinity purified anti-FDP antibody to at least one enzyme using cross-linking agents; and
immobilizing said at least one affinity purified anti-FDP antibody onto a solid phase.

48. The method of claim 47 wherein the antibodies are affinity purified polyclonal antibodies to FDP.

49. The method of claim 47 wherein the affinity purified polyclonal antibodies to FDP are immobilized onto plastic surface.

50. The method of claim 47 wherein the affinity purified polyclonal antibodies to FDP are labeled with an enzyme.

51. The method of claim 50 wherein the affinity purified polyclonal antibodies to FDP are labeled with horseradish peroxidase.

52. The method of claim 47 wherein the procedure is a sandwich type enzyme labeled immunoassay using microwell coated with antibodies to FDP and horseradish peroxidase conjugated antibodies to FDP.

53. The method of claim 47 wherein the procedure is a sandwich type enzyme labeled immunoassay using microwell coated with antibodies to FDP and horseradish peroxidase conjugated antibodies to fibrinogen.

54. The method of claim 52 is used to screen for lung cancer.

55. The method of claim 52 is used to screen for colon cancer.

56. The method of claim 52 is used to screen for ovarian cancer.

57. The method of claim 52 is used to screen for prostate cancer.

58. The method of claim 52 is used to screen for breast cancer.

59. The method of claim 52 is used to screen for liver cancer.

60. A method for producing an enzyme-linked immunosorbent assay for the monitoring of cancer treatment, said method comprising the steps of:
thiolating at least one affinity purified anti-FDP antibody;
isolating said at least one affinity purified anti-FDP antibody;
linking said at least one affinity purified anti-FDP antibody to at least one enzyme using cross-linking agents;
immobilizing said at least one affinity purified anti-FDP antibody onto a solid phase;
obtaining biological sample from a patient;
contacting said biological sample with said at least one affinity purified anti-FDP antibody immobilized onto said solid phase; and
measuring serum FDP level.

61. The method of claim 60 wherein the antibodies are affinity purified polyclonal antibodies to FDP.

62. The method of claim 60 wherein the affinity purified polyclonal antibodies to FDP are immobilized onto plastic surface.

63. The method of claim 60 wherein the affinity purified polyclonal antibodies to FDP are labeled with an enzyme.

64. The method of claim 63 wherein the affinity purified polyclonal antibodies to FDP are labeled with horseradish peroxidase.

65. The method of claim 60 wherein the procedure is a sandwich type enzyme labeled immunoassay using microwell coated with antibodies to FDP and horseradish peroxidase conjugated antibodies to FDP.

66. The method of claim 60 wherein the procedure is a sandwich type enzyme labeled immunoassay using microwell coated with antibodies to FDP and horseradish peroxidase conjugated antibodies to fibrinogen.

67. The method of claim 65 is used to monitor the treatment of lung cancer.

68. The method of claim 65 is used to monitor the treatment of colon cancer.

69. The method of claim 65 is used to monitor the treatment of ovarian cancer.

70. The method of claim 65 is used to monitor the treatment of prostate cancer.

71. The method of claim 65 is used to monitor the treatment of breast cancer.

72. The method of claim 65 is used to monitor the treatment of liver cancer.
